(19) United States
(12) Patent Application Publication
(10) Pub. No.: US 2005/0245971 A1
(43) Pub. Date: Nov. 3, 2005

(54) Implantable medical devices and related methods

(76) Inventors: Brian P. Brockway, Shoreview, MN (US); Perry A. Mills, Arden Hills, MN (US); Arthur J. Foster, Centerville, MN (US); Scott Lambert, East Bethel, MN (US); Kathy Lynn Sherwood, North Oaks, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/119,358

(22) Filed: Apr. 28, 2005

Related U.S. Application Data
(60) Provisional application No. 60/566,222, filed on Apr. 28, 2004.

(51) Int. Cl. A61N 1/00

(52) U.S. Cl. 607/2

(57) ABSTRACT

Implantable medical devices and associated methods are disclosed. In one implementation, the implantable medical device comprises a conductive housing and a remote electrode that is mechanically coupled to the conductive housing by a lead body. An amplifier is electrically connected to the remote electrode and the conductive housing for providing a signal representative of a voltage difference between the remote electrode and the conductive housing. In some methods in accordance with the present invention, the implantable medical device is implanted in an implant site overlaying one half of a rib cage of a human body. The implantable medical device produces a signal representative of the voltage difference between the remote electrode and the conductive housing and the signal is transmitted to a receiver located outside the human body.

### CROSS REFERENCE TO RELATED APPLICATION

The present application claims benefit under 35 U.S.C. §119(e) of U.S. Provisional Application No. 60/566,222, filed Apr. 28, 2004, which is hereby incorporated by reference.

### BACKGROUND OF THE INVENTION

Some types of implantable devices provide for measurement of ECG and other information which may be transmitted to an external recorder and/or analysis device. The information thus recorded can be used by a physician or other medical care provider to aid in diagnosis or treatment or for alerting emergency medical services of a life-threatening event. Current systems commercially available for the same or similar purpose include the Reveal® implantable loop recorder (ILR) available from Medtronic (Minneapolis, Minn.), animal monitoring devices available from Data Sciences International (St. Paul, Minn.), mobile outpatient cardiac telemetry systems and services available from Cardionet (San Diego, Calif.), and various hardwired systems.

The Medtronic Reveal is an ECG monitor intended for diagnosis of syncope or other rhythm disturbances. This device analyzes the ECG in real time. The device detects when a rhythm disturbance occurs and stores a segment of the ECG strip before and after the time of the rhythm disturbance. Issues with this include limited signal processing capability leading to poor detection accuracy. This device is often unable to, for example, detect atrial fibrillation accurately. In addition, it often falsely detects rhythm disturbances resulting in ECG's with no useful diagnostic utility filling the memory of the device. Memory in this device is limited to about 40 minutes, and the patient must visit the clinic in order for the memory of the device to be dumped and reset. Once the memory fills, a syncopal event can no longer be recorded. Since these events can occur very infrequently, this can limit the diagnostic utility of the device. The Reveal includes ECG electrodes that are incorporated into the body of the device. One electrode is in the header and the 2nd electrodes is an uninsulated portion located at the opposite end of the metallic body of the device.

The Data Sciences International (DSI) system for monitoring animals involves an implanted ECG, temperature, and pressure transmitter that telemeters a continuous ECG. Information from this device is transmitted in real time to a receiver. The receiver forwards a signal to a computing device where the signals are analyzed (ECGs for arrhythmias, intervals; pressure for systolic, diastolic, and mean pressure, heart rate, dP/dt, etc.) The transmitter employs flexible leads for sensing that extend from the body of the device.

The Cardionet system involves surface electrodes that are placed on the patient for monitoring ECG. The ECG signal is telemetered to a computing device that analyzes the ECG and identifies rhythm abnormalities. This device can forward a real time ECG to a monitoring station, or can notify the monitoring station if an abnormal rhythm is identified. This system packetizes the telemetered signal, incorporates time synchronization, and the receiver identifies whether a particular packet was received properly. If a packet was not received properly, the computing device signals to the transmitter to resend a packet. This device requires that surface electrodes be worn. Wires from the surface electrodes are connected to the telemetry device worn by the patient. This can particularly be a problem while the patient is sleeping. Also, since surface electrodes must be worn, patient compliance is an issue. Most patients are unwilling to wear surface electrodes for more than about three to four weeks. This system provides the advantage of real time monitoring can be accomplished. If the surface electrodes come loose, this can be identified immediately by the monitoring center and the patient can be contacted to reposition the electrodes.

Hardwired systems are available to serve this purpose. A computing device connects directly to surface electrodes for recording and/or analyzing ECG for the purpose of providing diagnostic information to the physician. These devices have no telemetry link and have the disadvantage that the patient must wear surface electrodes and be connected to the recorder. This can particularly be a problem while the patient is sleeping. Also, since surface electrodes must be worn, patient compliance is an issue. Most patients are unwilling to wear surface electrodes for more than about three to four weeks. Devices are often worn for two to four weeks. If problems have occurred in the recording, it will not be noticed for quite some time.

### BRIEF SUMMARY OF THE INVENTION

Implantable medical devices and associated methods are disclosed. In one implementation, the implantable medical device comprises a conductive housing and a remote electrode that is mechanically coupled to the conductive housing by a lead body. An amplifier is electrically connected to the remote electrode and the conductive housing for providing a signal representative of a voltage difference between the remote electrode and the conductive housing. In some methods in accordance with the present invention, the implantable medical device is implanted in an implant site overlaying one half of a rib cage of a human body. The implantable medical device produces a signal representative of the voltage difference between the remote electrode and the conductive housing and the signal is transmitted to a receiver located outside the human body.

### BRIEF DESCRIPTION OF THE DRAWING

FIG. 1 is a schematic illustration showing a system for monitoring one or more physiological signals telemetered from an implantable medical device implanted in a human patient.

FIG. 2 is a plan view showing an implantable medical device that is implanted in the body of a patient and a repeater that is supported by a lanyard that extends around the neck of the patient.

FIG. 3 is a plan view showing an implantable medical device that is implanted in a human body and a repeater that is supported by an elastic garment that extends about the human body.

FIG. 4 is an isometric view showing a portion of a human body with an implantable medical device implanted therein.

FIG. 5 is an isometric view showing a left implant site disposed in the left half of the human body shown in the previous figure.

FIG. 6 is an isometric view showing a right implant site disposed in the right half of the human body shown in the previous figure.

FIG. 7 is a transverse cross-sectional view of a human body with an implantable medical device implanted therein.

FIG. 8 is a cross-sectional view showing an implantable medical device in accordance with an exemplary embodiment of the present invention.

FIG. 9 is an additional cross sectional view of the implantable medical device shown in the previous figure.

FIG. 10 is an axial view of a lead assembly in accordance with an exemplary embodiment of the present invention.

FIG. 11 is a block diagram of an implantable medical device in accordance with an exemplary embodiment of the present invention.

FIG. 12 is a block diagram of an implantable medical device in accordance with an additional exemplary embodiment of the present invention.

FIG. 13 is a diagrammatic view of an implantable medical device in accordance with an exemplary embodiment of the present invention.

FIG. 14 is a schematic diagram showing an activity sensor and associated circuitry.

FIG. 15 is a diagrammatic view of an implantable medical device in accordance with an exemplary embodiment of the present invention.

FIG. 16 is a diagrammatic view of an implantable medical device in accordance with an exemplary embodiment of the present invention.

FIGS. 17A and 17B are diagram views showing a threading tool and a placement tool that may be employed to deploy an implantable medical device in accordance with the present invention.

FIGS. 18A-18C show electrodes incorporated into various portions of a housing of an implantable medical device.

FIG. 19 is a block diagram of an implantable medical device that is capable of producing a first signal that is representative of respiration and a second signal that is representative of ECG.

FIG. 20A and FIG. 20B show the recharging of an implantable medical device by transformer coupling energy from a recharging device located outside the body to a coil located inside the implantable medical device.

FIG. 21 is a block diagram showing an implantable medical device and a recharging device.

FIG. 22 is a diagrammatic view of an implantable medical device in accordance with an additional exemplary embodiment of the present invention.

FIG. 23 is a block diagram showing an implantable medical device and a recharging device that may be used to recharge the implantable medical device.

FIG. 24 is a block diagram showing an implantable medical device and a recharging device that may be used to recharge the implantable medical device.

FIG. 25 is a flowchart illustrating an exemplary method in accordance with the present invention.

FIG. 26 is a diagram view showing a placement tool and an associated method that may be employed to deploy an implantable medical device in accordance with the present invention.

FIG. 27 is an additional diagram view showing a placement tool and an associated method that may be employed to deploy an implantable medical device in accordance with the present invention.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

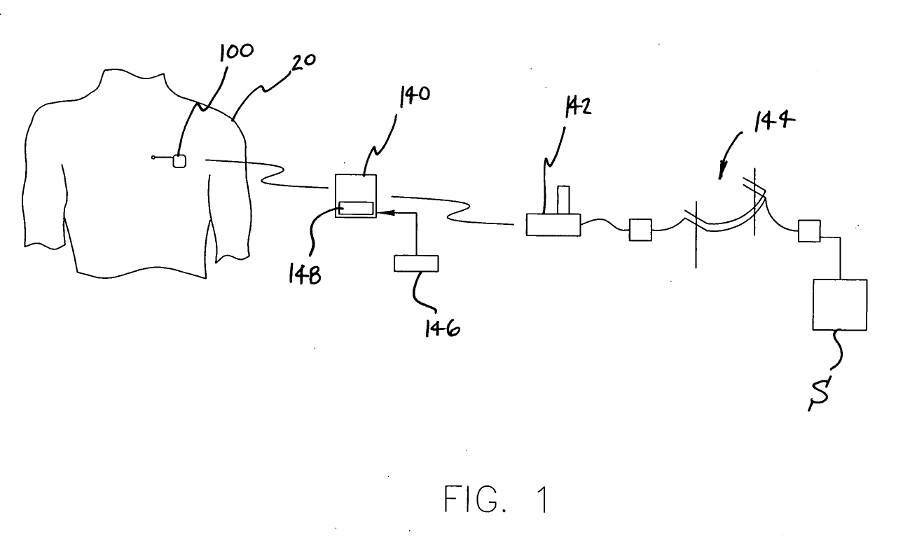

FIG. 1 is a schematic illustration showing a system for monitoring one or more physiological signals telemetered from implantable medical device 100 implanted in a human patient 20. In this illustrative embodiment, the system measures physiological signals such as ECG, pressure and/or temperature, and transmits (e.g., wirelessly) the waveforms of these signals to repeater 140 worn by or kept near patient 20. Repeater 140 receives the transmitted signals from implantable medical device 100 and retransmits (e.g., wirelessly) the signals to receiver/analyzer/storage buffer, RASB 142. Implantable medical device 100, repeater 140 and RASB 142 allow patient 20 to be monitored when lying in bed sleeping or going about normal daily activities. The RASB 142 may transmit the physiological data to a physician monitoring station S via a network 144. Network 144 may comprise various networks without deviating from the spirit and scope of the present invention. Examples of networks that may be suitable in some applications include the Internet and modem communication via telephone lines. Various communication techniques are described in the following U.S. Pat. Nos.: 5,113,869; 5,336,245; 6,409,674; 6,347,245; 6,577,901; 6,804,559; 6,820,057. The entire disclosures of the above-mentioned U.S. Patents are hereby incorporated herein by reference. Various communication techniques are described in the following U.S. patent applications: US2002/0120200 and US2003/0074035. The entire disclosures of the above-mentioned U.S. patent applications are also hereby incorporated herein by reference.

Implantable medical device 100 may be dedicated to patient monitoring, or it may alternatively include a therapeutic function (e.g., pacing, defibrillation, etc.) as well. Repeater 140 may comprise a barometric pressure sensor 146 that measures barometric pressure and communicates the measurement to computing device 148. Computing device 148 subtracts barometric pressure from pressure measured by implantable medical device 100 to provide a gauge pressure measurement of internal body pressure. This gauge pressure signal is then retransmitted by repeater 140 to RASB 142, or it may be communicated back to a medical device implanted in patient 20 to aid in controlling delivery of a therapy. The therapeutic function may be contained within a separate implantable device that is in communication with repeater 140 or/and implantable medical device 100. This therapeutic function may be controlled in part by information derived separately or in combination from repeater 140 or/and medical device.

Implantable medical device 100 may transmit signals in real time or pseudo real time (slightly delayed from real time). If the transmissions occur in true real time, and if the waveforms were to be transmitted either continuously or frequently, in order to achieve satisfactory battery life, the transmitter may employ a modulation scheme such as Pulse Interval Modulation (PIM) and use a relatively low transmit carrier frequency (for example, tens or hundreds of kHz). Another approach to conserving power might be to process the signals within the medical device to extract the useful information. If the volume of data comprising the useful information is much less than the signals from which it was derived, the useful information may then be stored for later transmission, or it may then be transmitted in real time or pseudo real time to a receiver located outside the body. One limitation that is apparent in the Medtronic REVEAL device (Minneapolis, Minn.) is that the device often fills memory with false positive strips of what it perceives to be aberrant rhythms. By transmitting the raw data to a processor located outside the body, the useful information contained in the signals can be more precisely extracted

A limitation of using PIM and a low carrier frequency is that the transmit range is relatively short and the signal transmission is subject to interference. This limitation can be overcome by locating repeater 140 in close proximity to implantable medical device 100. This can be accomplished by wearing repeater 140 in close proximity to implantable medical device 100 by attaching it to lanyard or clip, or by securing it to a strap or elastic garment worn on patient 20.

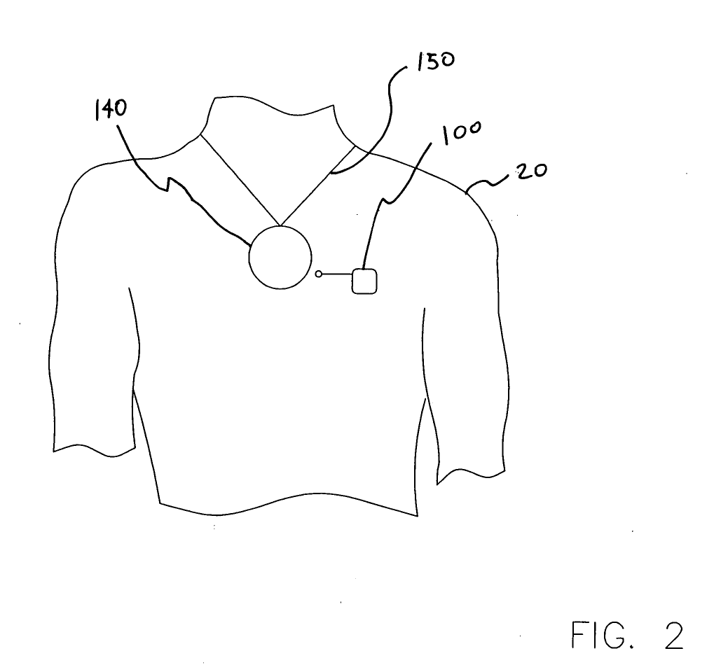

FIG. 2 is a plan view showing an implantable medical device 100 that is implanted in the body of a patient 20. A repeater 140 is supported by a lanyard 150 that extends around the neck of patient 20. Use of lanyard 150 allows repeater 140 to be carried in close proximity to implantable medical device 100.

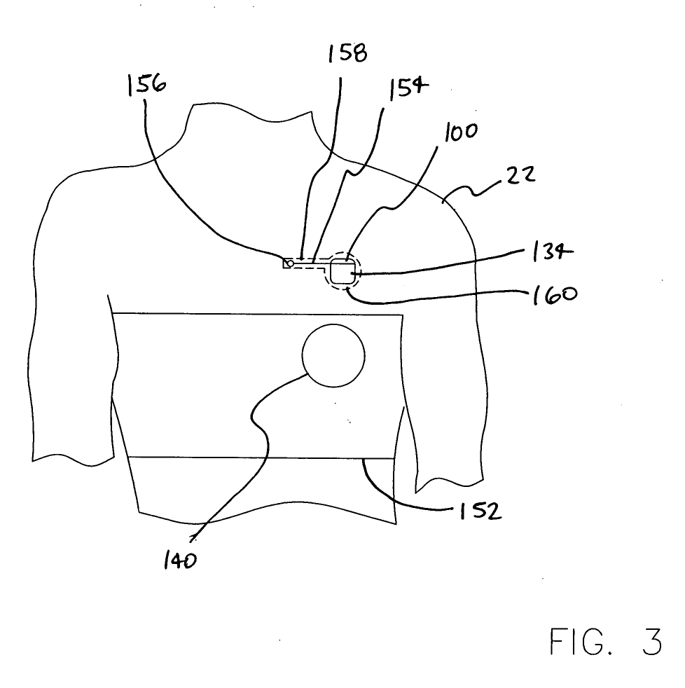

FIG. 3 is a plan view showing an implantable medical device 100 that is implanted in a human body 22. A repeater 140 is supported by an elastic garment 152 that extends about the human body 22. In the embodiment of FIG. 3, implantable medical device 100 comprises a housing 134, a lead body 154, and a remote electrode 156. With reference to FIG. 3, it will be appreciated that housing 134 is disposed in a pocket 160 that has been formed in the tissue of human body 22. With continuing reference to FIG. 3, it will be appreciated that remote electrode 156 is disposed in a channel 158 that has been formed in the tissue of human body 22.

In some methods in accordance with the present invention, pocket 160 and channel 158 are formed within a pre-selected implant site inside human body 22. Pocket 160 may be formed, for example, by making an incision with a cutting tool and pushing a blunt object through the incision to displace tissue and form pocket 160. For example, pocket 160 may be formed by pushing gloved fingers through the incision. Channel 158 may be formed, for example, by inserting a stylet into a lumen of lead body 154 and advancing lead body 154 into the body so that tissue is displaced and channel 158 is formed in the tissue. By way of a second example, channel 158 may be formed by inserting a groove director into pocket 160 and advancing the groove director into the body so that tissue is displaced and channel 158 is formed in the tissue. One groove director that may be suitable in some applications is commercially available from Universal Surgical Instruments of Glen Cove, N.Y., USA which identifies it by the part number 88-42-2695.

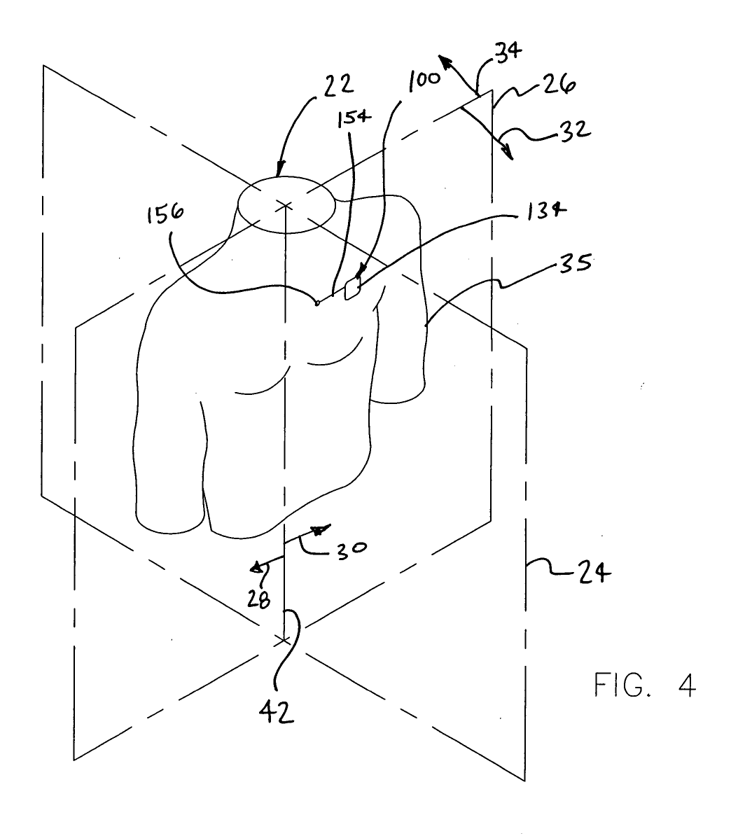

FIG. 4 is an isometric view showing a portion of a human body 22 with an implantable medical device 100 implanted therein. In FIG. 4, a central sagital plane 24 and a frontal plane 26 are shown intersecting human body 22. In the embodiment of FIG. 4, central sagital plane 24 and frontal plane 26 intersect one another at a median axis 42 of human body 22. With reference to FIG. 4, it will be appreciated that central sagital plane 24 bisects human body 22 into a right half 28 and a left half 30. Also with reference to FIG. 4, it will be appreciated that frontal plane 26 divides human body 22 into an anterior portion 32 and a posterior portion 34. In the embodiment of FIG. 4, central sagital plane 24 and a frontal plane 26 are generally perpendicular to one another.

With reference to FIG. 4, it will be appreciated that implantable medical device 100 is implanted in tissue proximate a left arm 35 of human body 22. In the embodiment of FIG. 4, implantable medical device 100 comprises a housing 134, a remote electrode 156 and a lead body 154 that mechanically couples remote electrode 156 to housing 134.

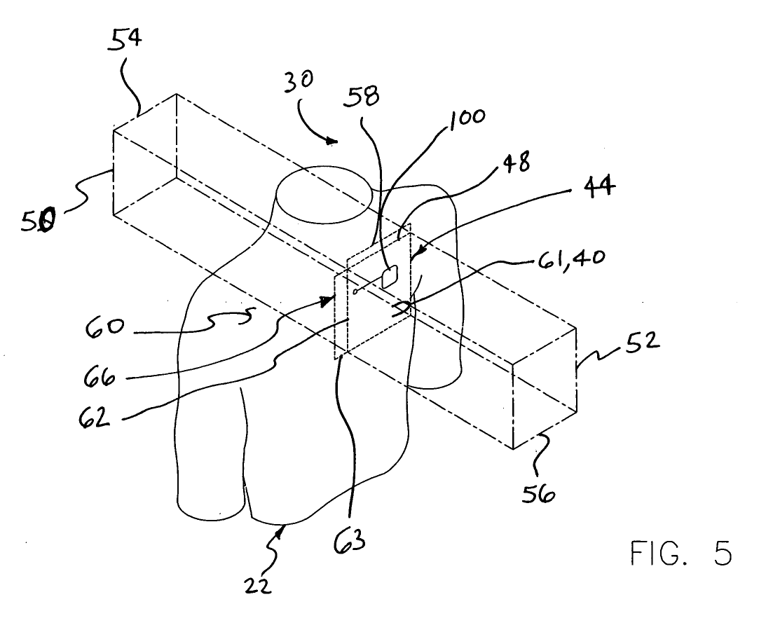

FIG. 5 is an isometric view showing a left implant site 44 disposed in the left half 30 of the human body 22 shown in the previous figure. With reference to FIG. 5, it will be appreciated that an implantable medical device 100 is disposed in the left implant site 44. As shown in FIG. 5, left implant site 44 may be defined by reference to a plurality of planes. A first sagittal plane 50 is shown contacting a left-most extent 62 of a sternum 66 of human body 22. A second sagittal plane 52 is shown contacting a left-most extent 61 of a rib cage 40. In the embodiment of FIG. 5, left implant site 44 extends laterally between first sagittal plane 50 and second sagittal plane 52. A superior transverse plane 54 is shown contacting a lower surface 48 of a left clavicle 58 of human body 22. An inferior transverse plane 56 is shown contacting a lower extent 63 of sternum 66. In the embodiment of FIG. 5, left implant site 44 extends between superior transverse plane 54 and inferior transverse plane 56. Some methods in accordance with the present invention, include the step of implanting implantable medical device 100 within left implant site 44. In some methods in accordance with the present invention, implantable medical device 100 is implanted between the skin 60 of the human body 22 and a front extent of rib cage 40.

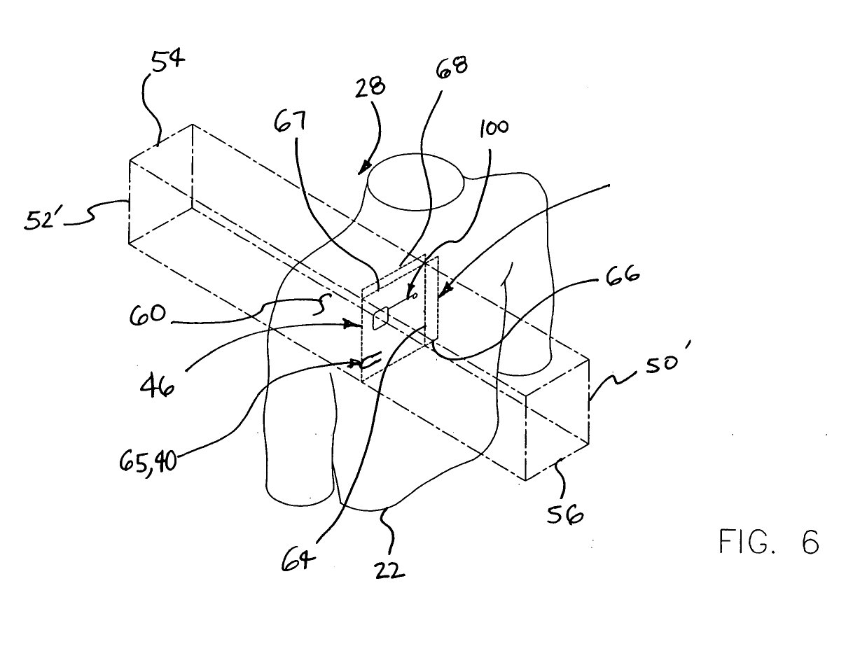

FIG. 6 is an isometric view showing a right implant site 46 disposed in the right half 28 of the human body 22 shown in the previous figure. With reference to FIG. 6, it will be appreciated that an implantable medical device 100 is disposed in the right implant site 46. As shown in FIG. 6, right implant site 46 may be defined by reference to a plurality of planes. A first sagittal plane 50′ is shown contacting a right-most extent 64 of a sternum 66 of human body 22. A second sagittal plane 52′ is shown contacting a right-most extent 65 of a rib cage 40. In the embodiment of FIG. 6, right implant site 46 extends laterally between first sagittal plane 50′ and second sagittal plane 52′. A superior transverse plane 54 is shown contacting a lower surface 67 of a right clavicle 68 of human body 22. An inferior transverse plane 56 is shown contacting a lower extent sternum 66. In the embodiment of FIG. 6, right implant site 46 extends between superior transverse plane 54 and inferior transverse plane 56. Some methods in accordance with the present invention, include the step of implanting implantable medical device 100 within right implant site 46. In some methods in accordance with the present invention, implantable medical device 100 is implanted between the skin 60 of the human body 22 and a front extent of rib cage 40.

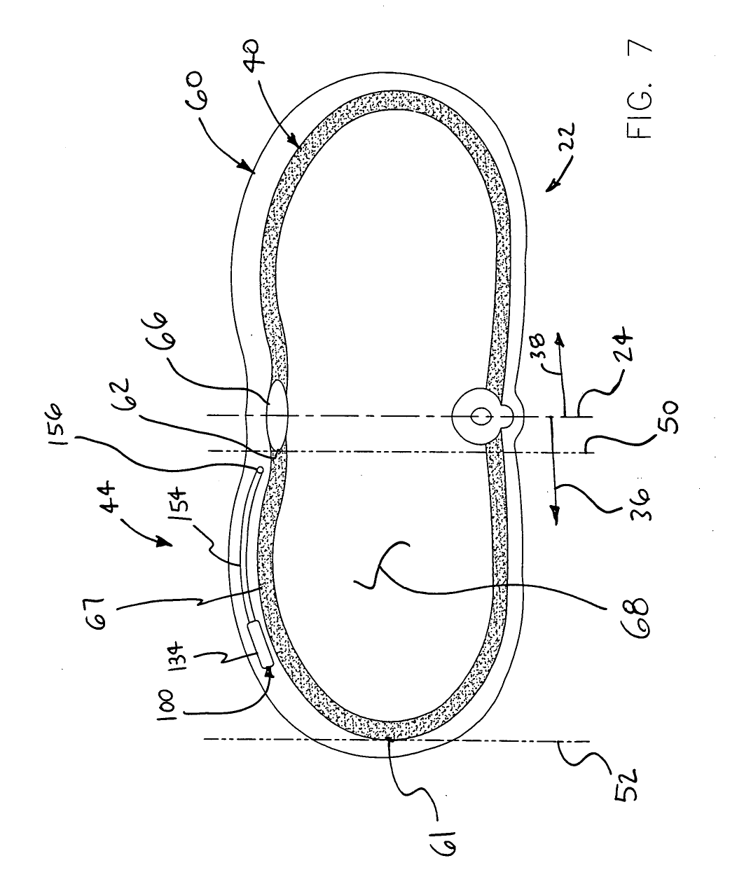

FIG. 7 is a transverse cross-sectional view of a human body 22 with an implantable medical device 100 implanted therein. The skin 60 and rib cage 40 of human body 22 are visible in this cross-sectional view. With reference to FIG. 7, it will be appreciated that implantable medical device 100 is disposed in a left implant site 44 of human body 22. Central sagital plane 24 is also shown in FIG. 7. With reference to FIG. 7, it will be appreciated that central sagital plane 24 bisects rib cage 40 into a right half 38 and a left half 36. With reference to FIG. 7, it will be appreciated that left implant site 44 generally overlays left half 36 of rib cage 40.

With reference to FIG. 7, it will be appreciated that implantable medical device 100 is disposed between skin 60 of human body 22 and a frontal extent 67 of the rib cage 40 of human body 22. In the embodiment of FIG. 7, left implant site 44 extends between a first sagittal plane 50 and a second sagittal plane 52. In FIG. 7, first sagittal plane 50 is shown contacting a left-most extent 62 of a sternum 66 of human body 22. Also in FIG. 7, second sagittal plane 52 is shown contacting a left-most extent 61 of rib cage 40.

In the embodiment of FIG. 7, implantable medical device 100 comprises a housing 134, a lead body 154, and a remote electrode 156. In FIG. 7, lead body 154 is shown assuming a generally curved shape. In some useful embodiments of the present invention, lead body 154 has sufficient lateral flexibility to allow lead body 154 to conform to the contour of left implant site 44. Also in some useful embodiments of the present invention, lead body 154 has sufficient lateral flexibility to allow lead body 154 to flex in compliance with muscle movements of human body 22. With reference to FIG. 7, it will be appreciated that lead body 154 does not extend into a chest cavity 68 of human body 20. Accordingly, it will be appreciated that lead 154 does not extend into a cavity of the heart of human body 20.

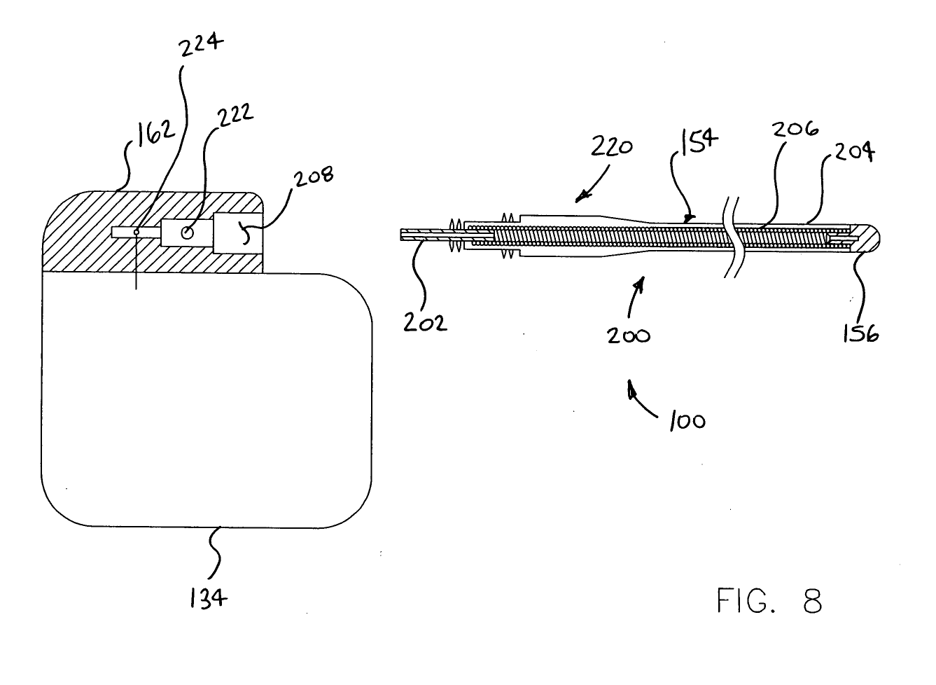

FIG. 8 is a cross-sectional view showing an implantable medical device 100 in accordance with an exemplary embodiment of the present invention. Implantable medical device 100 comprises a conductive housing 134, a header 162, and a lead assembly 200. Lead assembly 200 comprises a remote electrode 156 and a connector pin 202. Remote electrode 156 and connector pin 202 are mechanically coupled to one another by a lead body 154 of lead assembly 200. Lead body 154 comprises a coiled conductor 206 and an outer sheath 204. In some useful embodiments, outer sheath comprises a flexible material. Examples of flexible materials that may be suitable in some applications include silicone rubber and polyurethane.

Remote electrode 156 and connector pin 202 are also electrically connected to one another by coiled conductor 206. Coiled conductor 206 may comprise one or more filars wound in a generally helical shape. For example, coiled conductor 206 may comprise four helically wound filars. Remote electrode 156 may comprise various materials without deviating from the spirit and scope of the present invention. Examples of materials that may be suitable in some applications include stainless steel, Elgiloy, MP-35N, titanium, gold and platinum. Remote electrode 156 may also comprise a coating. Examples of coatings that may be suitable in some applications include carbon black, platinum black, and iridium oxide.

Header 162 defines a socket 208 that is dimensioned to receive a connecting portion 220 of lead assembly 200. Remote electrode 156 may be detachably attached to conductive housing 134 by inserting connecting portion 220 of lead assembly 200 into socket 208. In the embodiment of FIG. 8, a set screw 222 is disposed in a threaded hole defined by header 162. Set screw may be used to selectively lock connecting portion 220 of lead assembly 200 in socket 208. An electrical contact 224 is also shown in FIG. 8. Electrical contact 224 may make contact with connector pin 202 when connecting portion 220 of lead assembly 200 is disposed in socket 208.

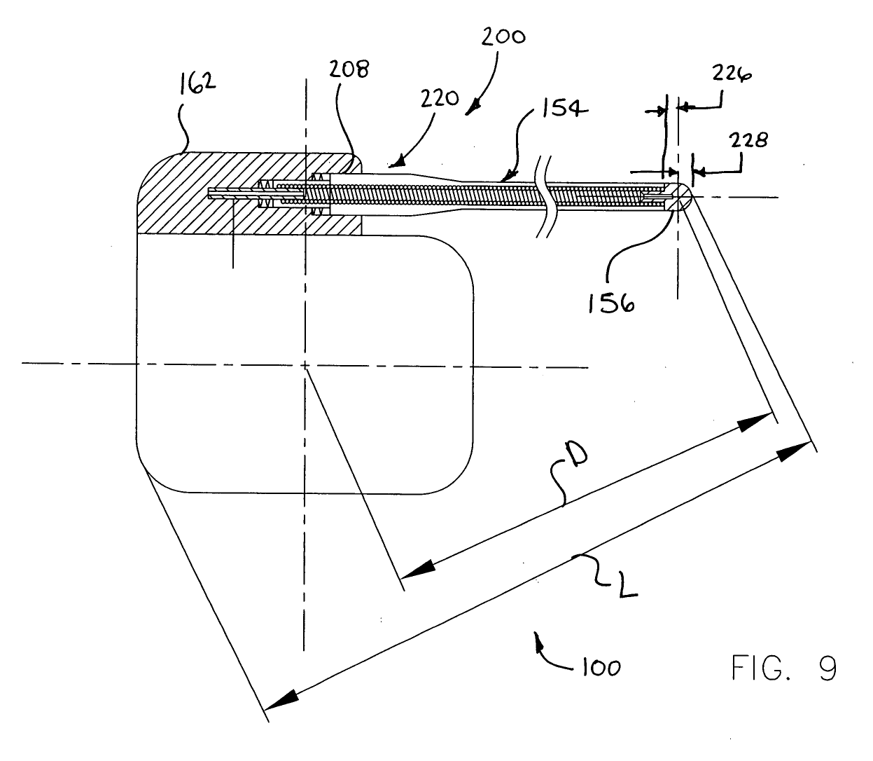

FIG. 9 is an additional cross sectional view of implantable medical device 100 shown in the previous figure. In the embodiment of FIG. 9, connecting portion 220 of lead assembly 200 is disposed in socket 208 defined by header 162. In the embodiment of FIG. 9, remote electrode 156 comprises a generally cylindrical body portion 226 having a generally circular lateral cross section. With reference to FIG. 9 it will be appreciated that remote electrode 156 also comprises a general rounded tip portion 228. In the embodiment of FIG. 9, tip portion 228 has a generally hemispherical shape.

With reference to FIG. 9, it will be appreciated that remote electrode 156 and lead body 154 are both free of anchors. In some applications, providing a remote electrode that is free of anchors may facilitate removal of the remote electrode from the human body. Additionally, providing a lead body that is free of anchors may facilitate removal of the lead from the human body.

With reference to FIG. 9, it will be appreciated that lead body 154 separates remote electrode 156 and conductive housing 134 by a center-to-center distance D. In some useful embodiments, distance D is selected to be relatively large so that a voltage differential between conductive housing 134 and remote electrode 156 is relatively large. In some useful embodiments of the present invention, distance D is greater than about 4.0 centimeters and less than about 10.0 centimeters. In some particularly useful embodiments, distance D is greater than about 5.0 centimeters and less than about 7.0 centimeters.

With continuing reference to FIG. 9, it will be appreciated that implantable medical device 100 has an overall length L. In some useful embodiments of the present invention, overall length L is selected so that conductive housing 134, remote electrode 156, and lead body 154 will all be received in an implant site overlaying one half of a rib cage of a human body. In some useful embodiments of the present invention, overall length L is greater than about 4.0 centimeters and less than about 13.0 centimeters. In some particularly useful embodiments, overall length L is greater than about 5.0 centimeters and less than about 10.0 centimeters.

Conductive housing 134 may comprise various materials without deviating from the spirit and scope of the present invention. Examples of materials that may be suitable in some applications include stainless steel, Elgiloy, MP-35N, titanium, gold and platinum. Conductive housing 134 may also comprise a conductive coating. Examples of conductive coatings that may be suitable in some applications include carbon black, platinum black, and iridium oxide. In the embodiment of FIG. 9, conductive housing 134 is free of insulating coatings so that the entire outer surface of conductive housing 134 is available to make electrical connection with body tissue. Embodiments of the present invention are possible in which a portion of conductive housing 134 is covered with an insulating coating, for example, PARYLENE.

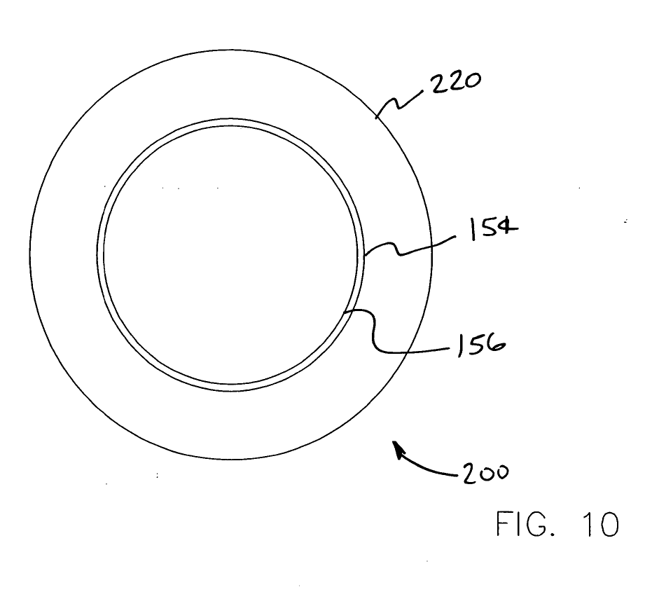

FIG. 10 is an axial view of lead assembly 200 shown in the previous figure. With reference to FIG. 10, it will be appreciated that remote electrode 156, lead body 154, and connecting portion 220 are all generally circular in cross section. In some applications, providing a remote electrode having a circular transverse cross-section may facilitate removal of the remote electrode from the human body. Additionally, providing a lead body having a circular transverse cross-section may facilitate removal of the lead from the human body.

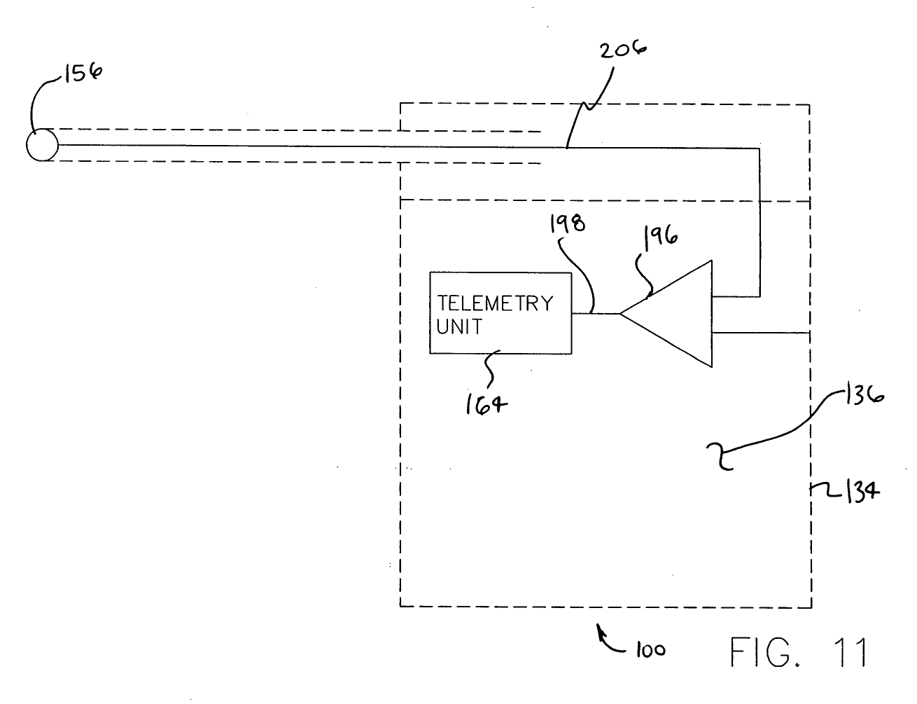

FIG. 11 is a block diagram of an implantable medical device 100 in accordance with an exemplary embodiment of the present invention. Implantable medical device 100 of FIG. 11 comprises a conductive housing 134 defining a cavity 136. In FIG. 11, an amplifier 196 is shown disposed in a cavity 136. A remote electrode 156 is electrically connected to amplifier 196 via a conductor 206. Amplifier 196 is also electrically connected to conductive housing 134. In the embodiment of FIG. 11, amplifier 196 is capable of detecting a voltage difference between conductive housing 134 and remote electrode 156. Amplifier 196 is also capable of producing a signal 198 that is representative of the voltage difference between conductive housing 134 and remote electrode 156. In FIG. 11, a telemetry unit 164 is shown connected to amplifier 196. In some useful embodiments of the present invention, implantable medical device 100 is disposed inside a human body and telemetry unit 164 is capable of transmitting signal 198 to a receiver located outside of the body.

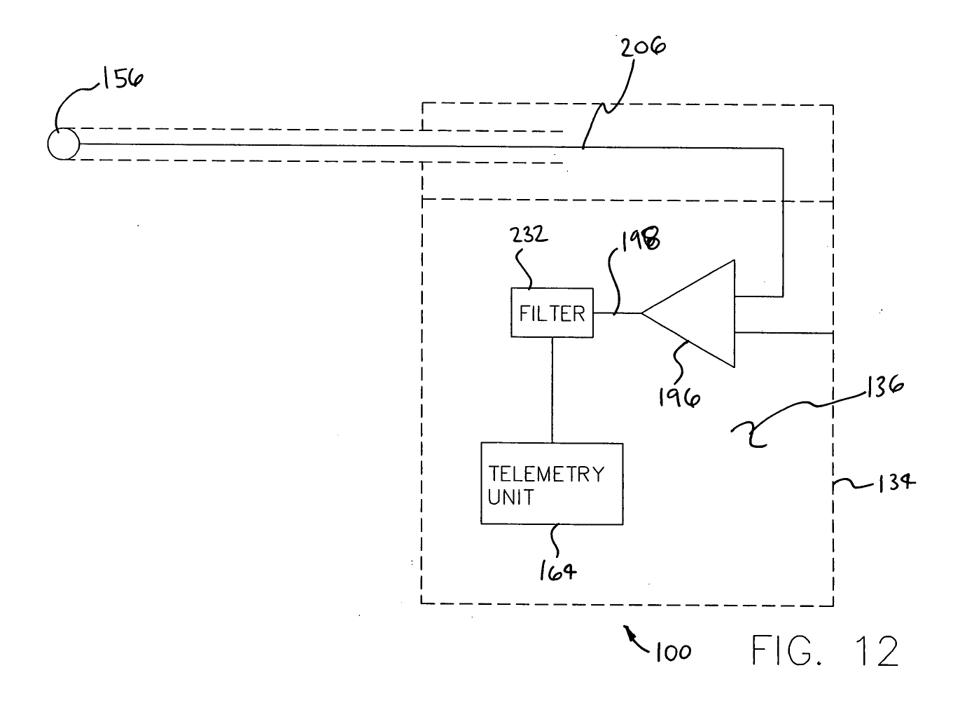

FIG. 12 is a block diagram of an implantable medical device 100 in accordance with an additional exemplary embodiment of the present invention. Implantable medical device 100 of FIG. 12 comprises a conductive housing 134 that is electrically connected to an amplifier 196. In the embodiment of FIG. 12, amplifier 196 is disposed within a cavity 136 defined by conductive housing 134. A remote electrode 156 is electrically connected to amplifier 196 via a conductor 206. In the embodiment of FIG. 12, amplifier 196 is capable of detecting a voltage difference between conductive housing 134 and remote electrode 156. Amplifier 196 is also capable of producing a signal 198 that is representative of the voltage difference between conductive housing 134 and remote electrode 156.

In the embodiment of FIG. 12, a filter 232 is electrically connected to amplifier 196. Filter 232 may be capable of filtering signal 198. Filter 232 may comprise, for example, a band-pass filter. When this is the case, filter 232 may pass a portion of signal 198 having frequency's between about 0.5 Hz and about 80.0 Hz. Filter 232 is electrically connected to a telemetry unit 164. In some useful embodiments of the present invention, implantable medical device 100 is disposed inside a human body and telemetry unit 164 is capable of transmitting at least a portion of signal 198 to a receiver located outside of the body.

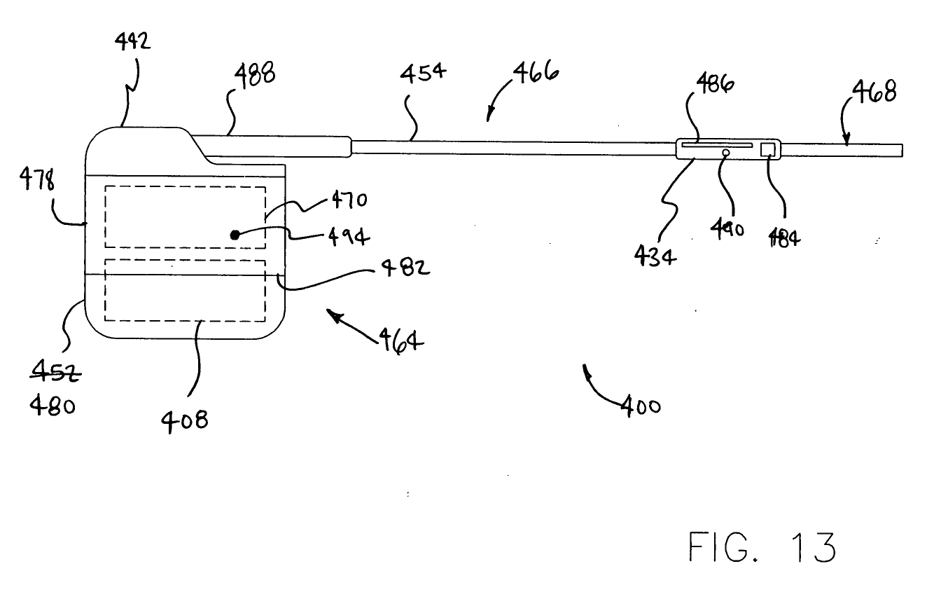

FIG. 13 is a diagrammatic view of an implantable medical device 400 in accordance with an exemplary embodiment of the present invention. Implantable medical device 400 may be used to measure a number of signals. In the embodiment of FIG. 13, for example, implantable medical device 400 is capable of measuring ECG, pressure, patient activity, patient posture, impedance, respiratory rate, respiratory effort, glucose, and temperature. In the embodiment of FIG. 13, implantable medical device 400 includes a telemetry unit 464 and remote sensing lead 466. Remote sensing lead 466 is capable of sensing pressure from an artery or vein, and communicating such signal to telemetry unit 464 for transmission. Remote sensing lead 466 may also contain one or more electrodes for sensing ECG as well as a pressure sensor.

Remote sensing lead 466 may employ one of a variety of pressure sensing means such as fiberoptic sensors, resonant sensor, piezoresistive sensors, capacitive sensors, and other sensors that can be fabricated in a diameter small enough to be safely introduced and reside within a vessel. In the preferred embodiment, the pressure sensing means may comprise a pressure transmission catheter (PTC 468), as described in U.S. Pat. No. 4,846,494 that can be introduced into an artery or vein. The entire disclosure of the above-mentioned U.S. patent is hereby incorporated by reference herein. The PTC approach as described in the '494 patent is advantageous in that it can be fabricated in a very small diameter. This is beneficial because the small size is less likely to damage the endothelial lining of the vessel and also because accidental pullout of the sensing catheter will result in far lesser complications.

PTC 468 refers the pressure signal to pressure sensor 484. Signal processing electronics 486 converts the signal from pressure sensor 484 to a signal that can be communicated to telemetry unit 464 via flexible lead body 454 and connector 488.

Remote sensing lead 466 may also incorporate a temperature sensor 490. Temperature sensor 490 would preferably be located within conductive housing 434 and the signal from temperature sensor 490 would be processed by signal processing electronics 486. The temperature signal would preferably be multiplexed with the pressure signal for communication to telemetry unit 464 via flexible lead body 454 and connector 488.

The housing of telemetry unit 464 may be constructed of three parts: a metallic portion 480 fabricated of a metallic material (e.g., titanium), an RF transparent portion 478 fabricated of ceramic, and a header 442. In the embodiment of FIG. 13, metallic portion 480 and RF transparent portion 478 are joined together at a seam 482. In FIG. 13, a battery 408 can be seen disposed in metallic portion 480.

Remote sensing lead 466 may also contain ECG sensing electrodes. In some embodiments, for example, conductive housing 434 of implantable medical device 400 may serve as one ECG sensing electrode while metallic portion 480 of the housing of telemetry unit 464 may serve as another ECG sensing electrode. Alternately, the second ECG sensing electrode could be incorporated into flexible lead body 454. This arrangement provides for sufficient spacing between the two ECG sensing electrodes to obtain adequate ECG signal amplitude and sensing of important features of the ECG such as p-waves for detection of atrial fibrillation. Flexible lead body 454 includes a conductor to connect the second ECG sensing electrode to signal processing electronics 486. The ECG signal is preferably multiplexed with the pressure and temperature signal for communication to telemetry unit 464 via flexible lead body 454 and connector 488.

Remote sensing lead 466 may further incorporate one or more conductors in flexible lead body 454 to serve as a transmitting and/or receiving antenna. Telemetry unit 464 may contain an activity sensor. The activity sensor may also comprise, for example, an accelerometer 494. As the patient moves about, g-forces placed on the accelerometer 494 by the patient may create an electrical signal that is representative of patient activity.

TU circuitry 470 contained in telemetry unit 464 is responsible for controlling power to remote sensing lead 466 and for transmitting the signals to repeater 440. In one exemplary embodiment, telemetry unit 464 has two operating states, on and off. When on, telemetry unit 464 transmits a PIM signal with a carrier frequency of about three hundred kHz. In another exemplary embodiment telemetry unit 464 compresses the signals to reduce the volume of data to be telemetered to reduce the power required by the transmitter. Power consumption can be further reduced by storing either the raw or compressed data in memory for a period of time, a few seconds for example, and then transmitting data at multiples of real time to repeater 440 or to RASB 442. In this approach, the transmitter is a high frequency transmitter operating at about nine hundred MHz, for example. Although such a high frequency transmitter consumes significantly more power when operating, it also provides for a much faster data transmission rate and therefore needs to operate for a much shorter period of time. It therefore allows several seconds of data stored in memory to be transmitted in a fraction of a second. Such an approach also allows the transmitter to employ more reliable communication means. For example, instead of using PIM, this approach allows for the use of frequency shift keying (FSK) modulation, a more robust modulation scheme compared to PIM. Further, transmitted data can be divided into packets and error correction codes (ECC) can be added to each packet. When a transmitted data packet is received at RASB 442, the ECC can be evaluated to determine if the packet was received correctly. RASB can either ignore such a corrupt packet, or it can be equipped with bi-directional communication such that it signals back to implantable medical device 400 that the packet was not received correctly and request that it be retransmitted by implantable medical device 400.

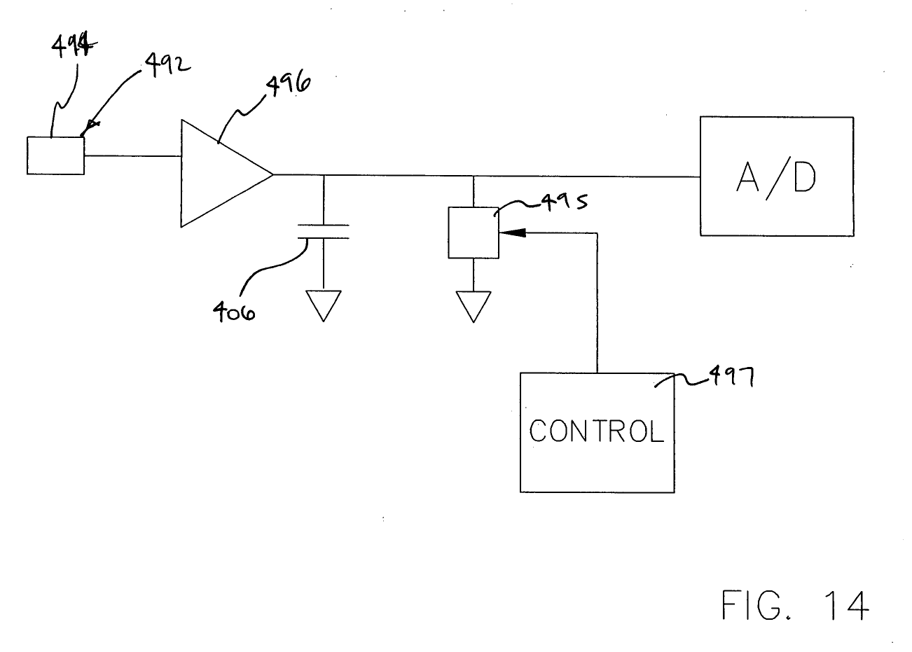

FIG. 14 is a schematic diagram showing an activity sensor 492 and associated circuitry. Telemetry unit 464 (shown in the previous figure) may contain activity sensor 492 and it's associated circuitry. Activity sensor 492 may comprise, for example, an accelerometer 494. As the patient moves about, g-forces placed on the accelerometer 494 by the patient's movement create an electrical signal that is amplified by an amplifier 496. The output of amplifier 496 is a current source that charges capacitor 406 with a fixed amount of charge. Once that level of charge is reached, a pulse is triggered and the charge on capacitor 406 is dumped, indicating that a quantum of patient activity has occurred. Pulses are counted over a unit time, a few minutes for example, to indicate the degree of patient activity. In the embodiment of FIG. 14, a switch 495 and a controller 497 cooperate to dump the charge on capacitor 406.

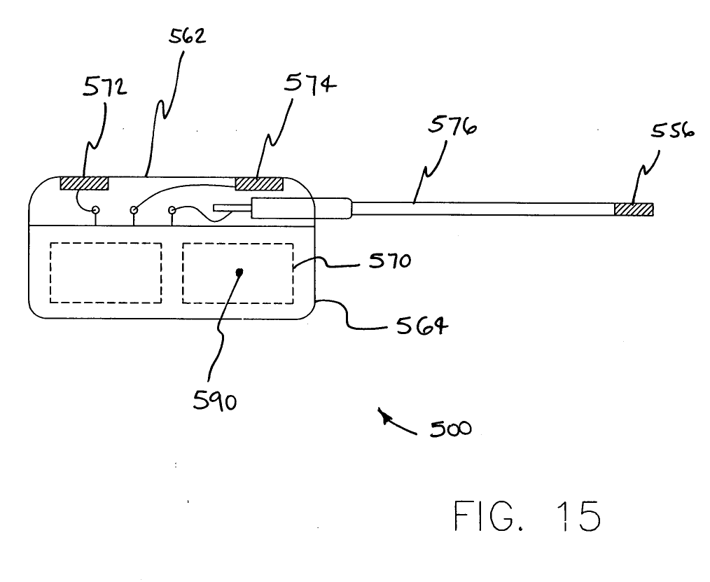

FIG. 15 is a diagrammatic view of an implantable medical device 500 in accordance with an additional exemplary embodiment of the present invention. In the embodiment of FIG. 15, implantable medical device 500 is used to monitor ECG, activity, and temperature. In this embodiment, since pressure is not necessarily being measured, the need for a remote sensing lead including a pressure sensor is eliminated. A temperature sensor 590 is contained within telemetry unit 564. Telemetry unit 564 includes TU circuitry 570. Data transmission approaches in this embodiment are similar in function to those previously described.

In the embodiment of FIG. 15, a first ECG electrode 572 and a second ECG electrode 574 are integral to header 562. A remote electrode 556 is contained at the distal end of flexible lead 576. Flexible lead 576 allows for remote electrode 556 to be directed to a site at the time of implantation that allows for a high quality ECG. By proper placement of telemetry unit 564 under the skin, it is possible to obtain two ECG channels using remote electrode 556 as a common electrode, allowing for measurement of two different ECG vectors. Further, if implantable medical device 500 were only capable of transmitting a single ECG channel, remote electrode 556 could be selectively paired by TU circuitry 570 to serve as a common electrode for either first ECG electrode 572 or second ECG electrode 574. This would allow fine-tuning of the ECG signal following implantation via a programmable function incorporated into TU circuitry 570. Such fine-tuning would allow the physician to select that electrode pair that provided, for example, the highest amplitude p-wave, or the least amount of muscle noise. Flexible lead 576 could also incorporate additional conductive elements to accommodate a transmitting and/or receiving antenna for the transmitter contained in telemetry unit 564.

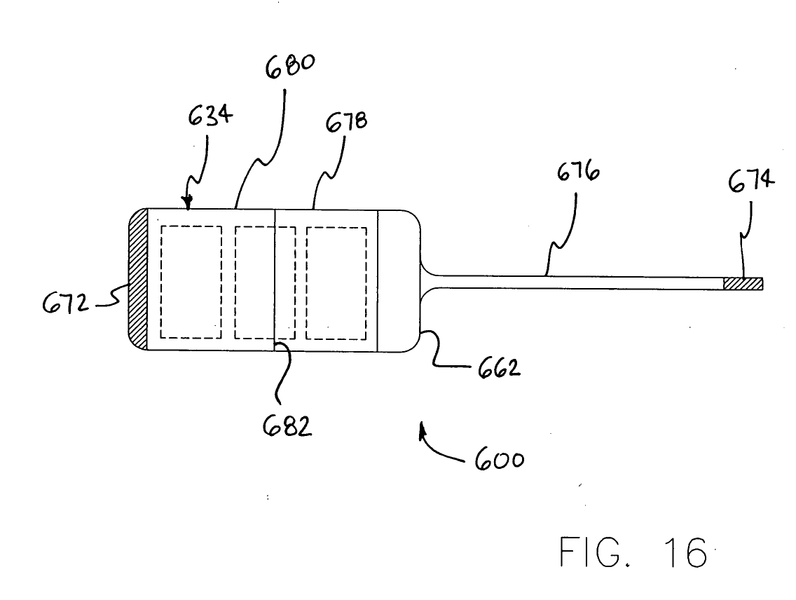

FIG. 16 is a diagrammatic view of an implantable medical device 600 in accordance with an additional exemplary embodiment of the present invention. In the embodiment of FIG. 16, implantable medical device 600 comprises a first electrode 672 and a second electrode 674. By placing one electrode at the distal end of flexible lead 676, sufficient spacing can be obtained between the two electrodes to detect a good quality ECG signal. In addition, placing an electrode on the end of flexible lead 676 provides for a greater degree of flexibility in placement of the electrodes relative to each other. This has the potential to improve the diagnostic quality of the ECG vector because flexibility in positioning could allow the physician to adjust the relative location of electrodes to improve the amplitude of the p-wave, t-wave, or other clinically significant features of the ECG waveform. The housing 634 of implantable medical device 600 may be constructed of three parts: a metallic portion 680 fabricated of a metallic material (e.g., titanium:; an RF transparent portion 678 fabricated of ceramic, and a header 662. The metallic portion 680 and RF transparent portion 678 are joined together at a seam 682. Metallic portion 680 is electrically insulated with parylene, except for the portion comprising first electrode 672. Flexible lead 676 may extend approximately four to ten centimeters distal to header 662.

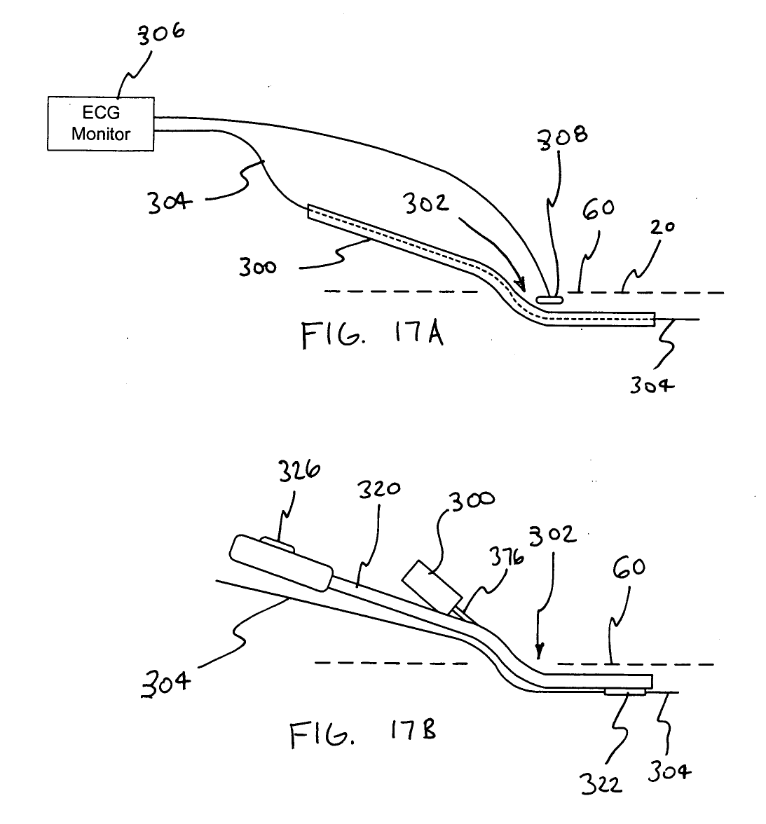

FIGS. 17A and 17B are diagram views showing a threading tool 300 and a placement tool 320 that may be employed to deploy an implantable medical device 300 in accordance with the present invention. To implant implantable medical device 300, an incision 302 is made where the device is to be inserted under the skin 60 of patient 20 and a pocket is formed under the skin 60 distal to the incision to accommodate the housing of implantable medical device. Threading tool 300 has a hollow lumen and is directed through the incision 302 and under the skin 60 to the desired location for a remote electrode of the implantable medical device. Once in location, a guidewire 304 is inserted into the lumen and threading tool 300 is extracted. Guidewire 304 is electrically insulated with the exception of a distal portion thereof. To evaluate a location for the remote electrode of the implantable medical device, the proximal end of guidewire 304 can be connected to an ECG monitoring instrument 306 while the other input to the ECG monitoring instrument 306 is connected to a temporary electrode 308 placed in the incision 302 at the approximate location where housing of the implantable medical device will be placed when the implantable medical device is implanted. If the result is satisfactory, the housing of the implantable medical device 300 and the flexible lead 376 of the implantable medical device 300 are attached to a placement tool 320. Placement tool 320 contains a guide 322 through which guidewire 304 is inserted. Placement tool 320 is then directed along guidewire 304 until guide 322 has reached the end of guidewire 304. Release 326 is then triggered, the housing of implantable medical device 300 and flexible lead 376 from placement tool 320. Placement tool 320 may then be extracted, leaving the housing of implantable medical device 300 and flexible lead 376 in position. The housing of implantable medical device 300 is positioned within the pocket adjacent to the incision and the incision is closed.

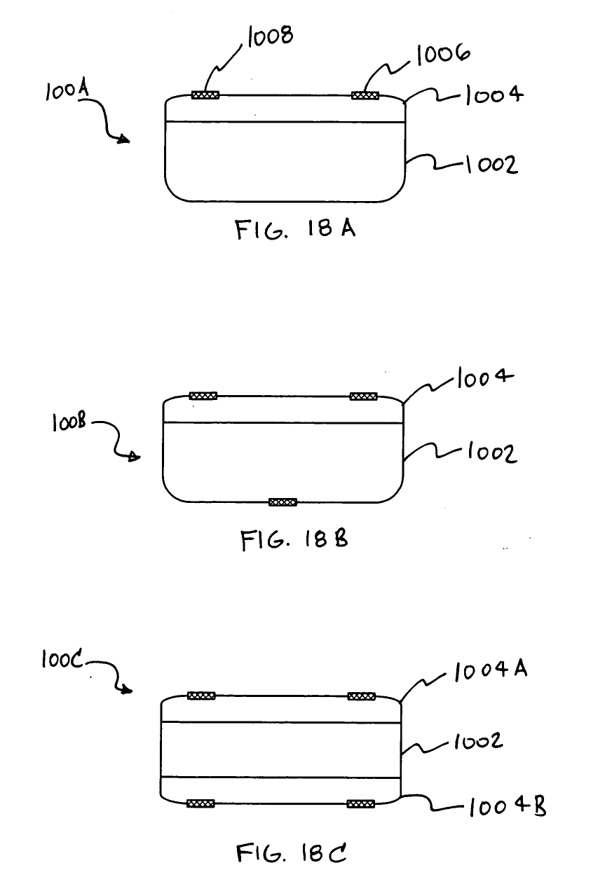

Various alternative lead-less embodiments of implantable medical device 100 are contemplated. For example, as shown in FIGS. 18A-18C, electrodes may be incorporated into various portions of the housing of the device 100. In each of these embodiments, the housing may include a case portion 1002 made of ceramic for example, and header portion 1004 made of a polymeric material. The electrodes 1006, 1008, 1010, 1012 may comprise a conductive material embedded in the header 1004 and/or case 1002, and the orientation of the electrodes and the distance between the electrodes may be maintained by the non-conductive portions of the housing, such as the ceramic case 1002 and/or the polymeric header 1004, in order to fix orientation for best signal capture. The housing holds and orientates one or more sensing electrodes 1006, 1008, 1010, 1012 for purposes of measuring ECG signals or other bio-potential signals such as EEG, EMG, ECG etc., or respiratory effort and/or cardiac stroke volume via impedance. These signals may be transmitted and or recorded as described previously.

The electrodes 1006, 1008, 1010, 1012 may be made from any suitable sensor electrode material (e.g., Stainless Steel, Elgiloy, MP-35N, Titanium, etc.) and may be coated to increase sensing capability (i.e.: carbon black, platinum black, iridium oxide, etc). The electrode surface may be smooth or porous coated, again to increase sensing capability. The electrodes may be located in-line or orthogonally opposed to increase the relative distance between them for improved capability. The macroscopic surface area of each electrode may vary depending on the application and the microscopic surface finish. The electrodes may be disposed in or on (e.g., embedded or coated) the header 1004 or the case 1002, and provided that they remain electrically isolated from each other and the rest of the structure. This may be accomplished by fabricating the case 1002 and/or header 1004 of a non-conductive material, or if a conductive material is used for the case 1002, by isolating the electrodes from the case with an insulating material.

A single header arrangement may be used as shown in FIGS. 18A and 10B, or a double header arrangement may be used as shown in FIG. 18C. With any one of these arrangements, two, three, four or more electrodes may be used depending on the number of electrical channels the device electronics allows for, the surface area required for each electrode and the signal to be measured in a given application. Additional electrodes may be provided via a flexible or semi-flexible wire lead arrangement as described previously herein, which would allow for further electrode spacing for increased signal resolution.

For measuring respiratory effort/respiratory rate, a constant current carrier signal may be injected between two electrodes. The carrier signal may be amplitude modulated by the changing impedance between the electrodes due to respiratory effort. The amplitude modulated signal may be demodulated and band-passed filtered for respiratory signals producing a changing voltage proportional to respiratory effort which can then be transmitted and or recorded. Cardiac stroke volume can be attained using similar methods but with a band pass tailored to the cardiac signal. An intra-cardiac electrode as one of the electrodes in the configuration would provide an improved measurement of cardiac stroke volume. Each of these techniques could be accomplished using a four electrode method, as Well, with one electrode pair providing the constant current, and another electrode pair to provide the measurement. This results in a more accurate measurement by eliminating the electrode impedance. All four electrodes could be configured in the header of the device, in the body of the device, via a flexible or semi-flexible wire arrangement, or in any combination of these electrode types.

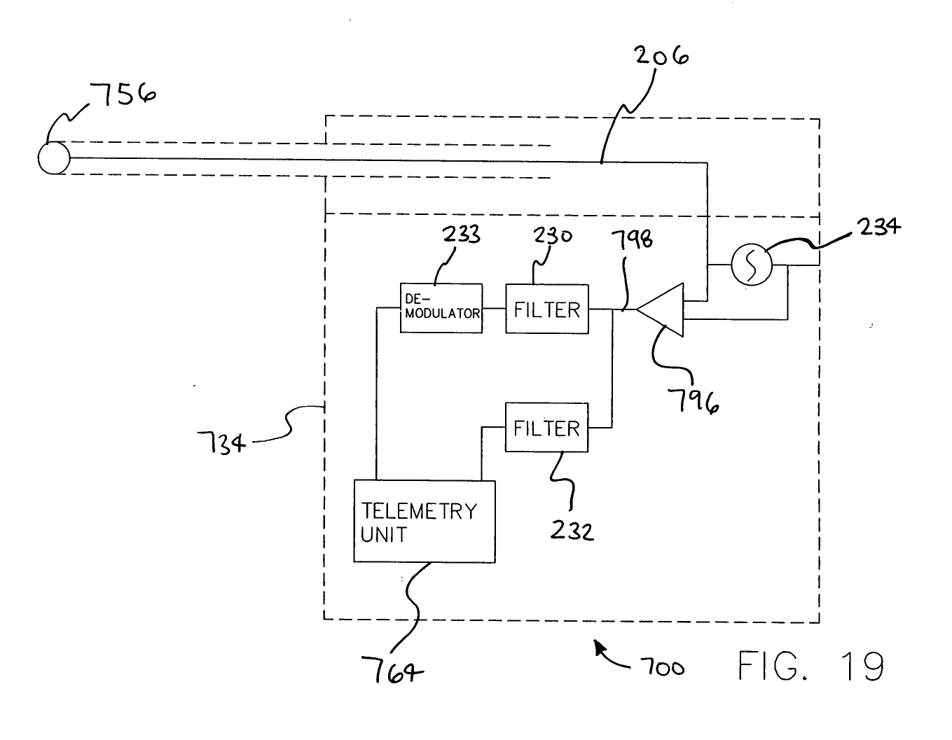

FIG. 19 is a block diagram of an implantable medical device 700 that is capable of producing a first signal that is representative of respiration and a second signal that is representative of ECG. Implantable medical device 700 of FIG. 19 comprises a conductive housing 734 that is electrically connected to a current source 234. A remote electrode 756 is also electrically connected to current source 234 via a conductor 206. In the embodiment of FIG. 19, current source 234 provides a substantially constant current traveling between conductive housing 734 and remote electrode 756.

In the embodiment of FIG. 19, an amplifier 796 is arranged to detect a voltage difference between conductive housing 734 and remote electrode 756. Amplifier 796 is also capable of producing a signal 798 that is representative of the voltage difference between conductive housing 734 and remote electrode 756. In the embodiment of FIG. 19, a first filter 230 and a second filter 232 are both connected to amplifier 796.

First filter 230 may comprise, for example, a band-pass filter that passes a portion of signal 798 that is related to the respiration of a human patient. For example, first filter 230 may pass a portion of signal 798 having frequency's between about 0.2 Hz and about 2.0 Hz. A de-modulator 233 is provided for demodulating the respiration related portion of signal 798.

Second filter 232 may comprise, for example, a band-pass filter that passes a portion of signal 798 that is related to ECG. For example, second filter 232 may pass a portion of signal 798 having frequency's between about 0.2 Hz and about 80.0 Hz. First filter 230 and second filter 232 are both electrically connected to a telemetry unit 764. In some useful embodiments of the present invention, implantable medical device 700 is disposed inside a human body and telemetry unit 764 is capable of transmitting at least a portion of signal 798 to a receiver located outside of the body.

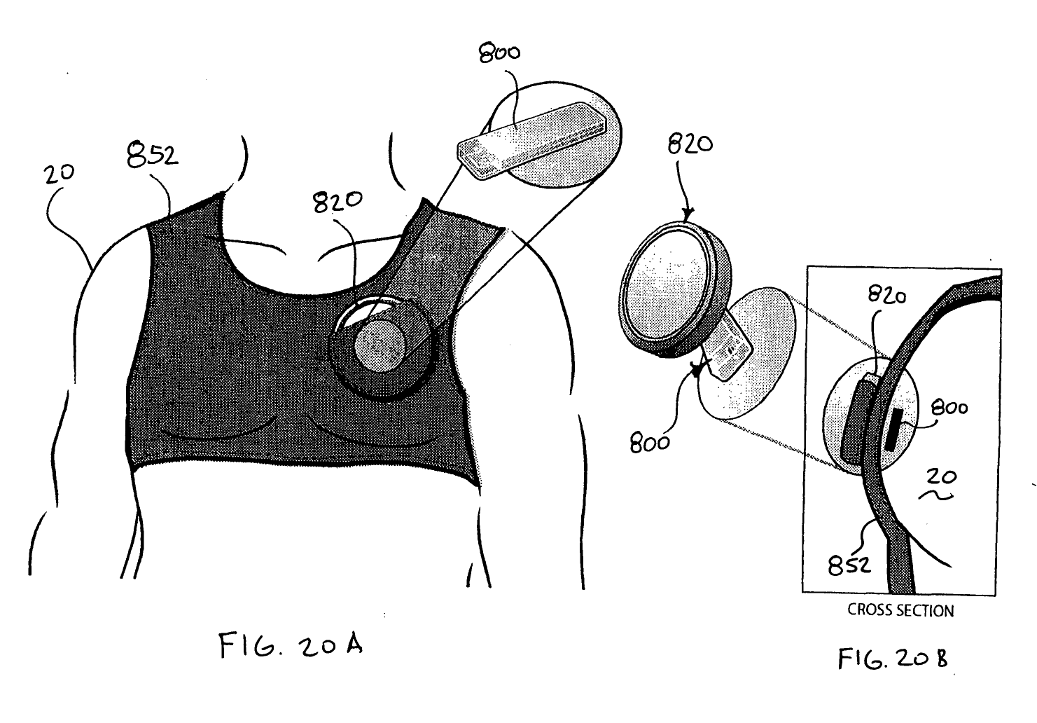

To extend the useful life, an implantable medical device 800 in accordance with the present invention may contain a rechargeable battery. As shown in FIG. 20A and FIG. 20B, recharging may be performed by transferring energy into implantable medical device 800 by transformer coupling energy from a recharging device 820, located outside the body, to a coil located in implantable medical device 800. The secondary of the transformer coil, located in implantable medical device 800, would drive circuitry that would create a charging current for the rechargeable battery.

For convenience, the charging device may be battery powered and portable and could be worn by patient 20 in an elastic garment 852 when necessary for recharging. The use of an elastic garment 852 would assure the device were held stably in proper position for charging. Alternately, recharging device 820 could contain a replaceable adhesive surface such that it could be located on the skin in close proximity to implantable medical device 800. In order to make it easy for the patient to place the recharging device properly, an indicator would tell the patient when the device was aligned properly, as measured by current being transferred into implantable medical device 800. A second indicator may tell the patient when the rechargeable battery is fully charged based on information transmitted from the implantable device to the recharging device.

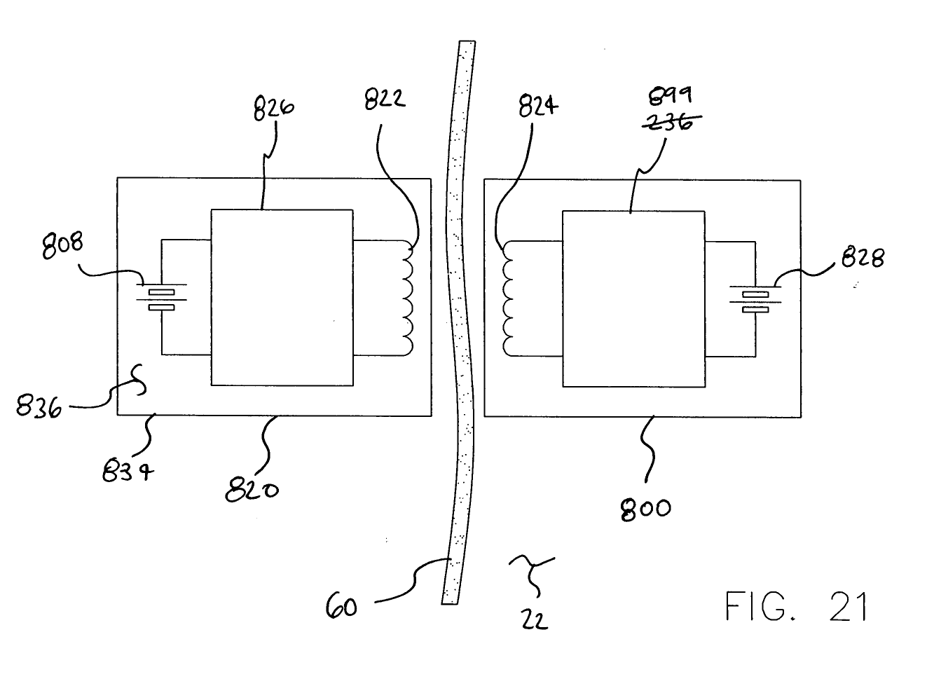

FIG. 21 is a block diagram showing an implantable medical device 800 and a recharging device 820. In the embodiment of FIG. 21, implantable medical device 800 is disposed inside a human body 22 and recharging device 820 is disposed outside of the human body 22. The skin 60 of the human body 22 is shown extending between implantable medical device 800 and recharging device 820 in FIG. 21.

In the embodiment of FIG. 21, recharging device comprises a first coil 822 and a first battery 808 coupled to first coil 822 for exciting first coil 822. A control circuit 826 is connected between first coil 822 and first battery 808. Control circuit 826 is capable of generating the oscillating current necessary to inductively couple first coil 822 of recharging device 820 with a second coil 824 of implantable medical device 800.

Implantable medical device comprises a second battery 828 and a second coil 824 coupled to second battery 828 for charging second battery 828. A charging circuit 899 is connected between second coil 824 and second battery. Charging circuit 899 may comprise, for example, a voltage regulator that is capable of controlling the magnitude of the voltage that is applied to second battery 828 during charging. Charging circuit 899 may also comprise, for example, a current regulator that is capable of controlling the magnitude of the current that is applied to second battery 828 during charging.

In the embodiment of FIG. 21, first coil 822 and second coil 824 are inductively coupled to one another so that second battery 828 is charged while first battery 808 is depleted. With reference to FIG. 21, it will be appreciated that recharging device 820 comprises a housing 834 defining a cavity 836. In the embodiment of FIG. 21, first battery 808 is disposed within cavity 836 defined by housing 834. In some useful embodiments of the present invention, first battery 808 is capable of satisfying the power requirements of recharging device 820. For example, first battery 808 may have sufficient capacity to fully charge second battery 828 and while, at the same time, compensating for energy lost during the charging of the second battery. In such embodiments, first battery 808 may be larger than second battery 828. Also in such embodiments, first battery 808 may be the sole source of power for recharging device 820. This arrangement may allow the user of implantable medical device to remain ambulatory during the charging process.

Various charging techniques are described in the following U.S. Pat. Nos.: 3,454,012; 3,824,129; 3,867,950; 3,492,535; 4,014,346; 4,057,069; 4,082,097; 4,096,866; 4,172,459; 4,441,210; 4,562,840; 4,679,560; 4,741,339; 5,279,292; 5,350,413; 5,411,537; 5,690,693; 5,702,431; 5,991,665; 6,067,474; 6,154,677; 6,324,431; 6,505,077; 6,516,227; 6,549,807; and 6,850,803. The entire disclosures of the above-mentioned U.S. Patents are hereby incorporated herein by reference.

In another embodiment, battery 828 of implantable medical device 828 may be recharged by deriving power from an implanted power source. Such an implanted power source may derive power from a human body by mechanical, thermal and/or chemical means. Examples of implantable power sources that derive power from a human body by thermal means include those described in U.S. Pat. No. 6,470,212 and U.S. Pat. No. 6,640,137. Examples of implantable power sources that derive power from a human body by mechanical means include those described in U.S. Pat. Nos. 3,943,936; 5,431,694; and 6,822,343 and U.K. Patent Application Number GB 2350302. The entire disclosure of each of the above-mentioned patents and patent application is hereby incorporated by reference herein. The implantable power source may be connected to charging circuit 899 and/or second battery 828 by a first wire and a second wire.

In some useful embodiments of the present invention, implantable medical device 800 may include a charge counter to track the amount of charge that has been consumed from the battery. In addition, implantable medical device 800 also incorporates a counter to track the amount of charge that has been depleted from battery 828. By tracking charge added and charge depleted, remaining battery life can be determined and communicated to an external receiver. When battery 828 is fully charged, both the charge added and charge depleted counters are reset to zero. The circuits used to count charge have some inherent error. If this error were allowed to accumulate through multiple charges and discharges of battery 828, the remaining charge in the battery as indicated by the charge added and charge depleted counters battery life indicator may have limited value. To address this problem, implantable medical device 800 contains a circuit that measures charging current to battery 828. When the charging current present indicates that battery 828 has reached full charge, both the charge depleted and charge added counters are reset.

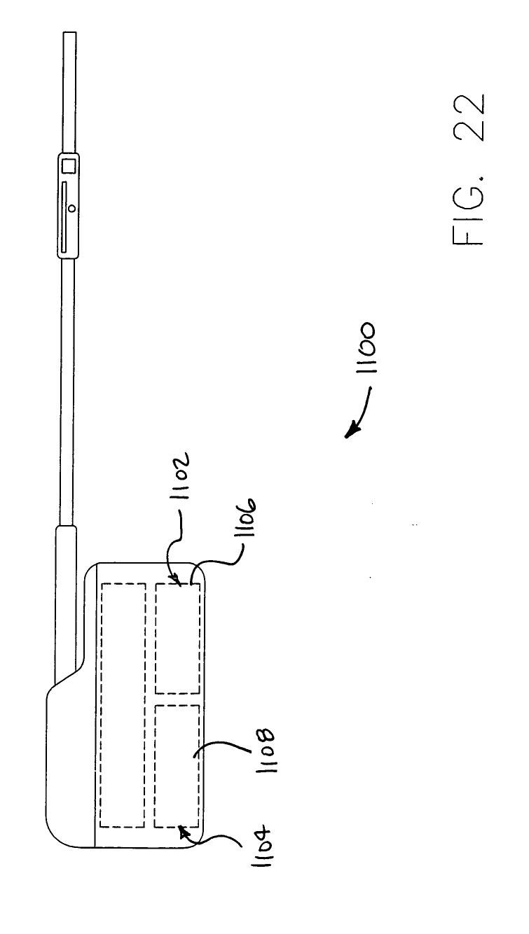

FIG. 22 is a diagrammatic view of an implantable medical device 1100 in accordance with an additional exemplary embodiment of the present invention. Implantable medical device 1100 comprises a first energy storage element 1102 and a second energy storage element 1104. In the embodiment of FIG. 22, first energy storage element 1102 comprises a capacitor 1106 and second energy storage element 1104 comprises a battery 1108. In the embodiment of FIG. 22; implantable medical device 1100 employs a first energy storage element 1102, such as capacitor 1106, that can store a smaller amount of charge than can be stored in battery 1108, but can store charge at a much faster rate than battery 1108. By placing a charging device near implantable medical device 1100 for a short period of time, first energy storage element 1102 is fully charged. Once first energy storage element 1102 is fully charged, additional charge coupled into implantable medical device 1100 from the charging device may be directed toward charging battery 1108. Once the charging device is pulled away and is no longer coupling energy into implantable medical device 1100, the charge stored in first energy storage element 1102 is transferred into battery 1108.

This architecture, employing a fast charging element and a slower charging element (e.g., a battery) may have advantages in certain situations. For example, suppose that battery 1108 had a charge capacity equal to about one hundred and fifty days of operation of implantable medical device 1100 and first energy storage element 1102 had a capacity of about seven days of operation. Normal charging time for battery 1108 may be about two hours, while charge time for first energy storage element 1102 was only about thirty seconds. In this scenario, the patient could obtain a charge equal to about one full week of operation in about thirty seconds. Many patients may find this protocol more convenient than wearing a vest holding a recharging device for two hours every three months.

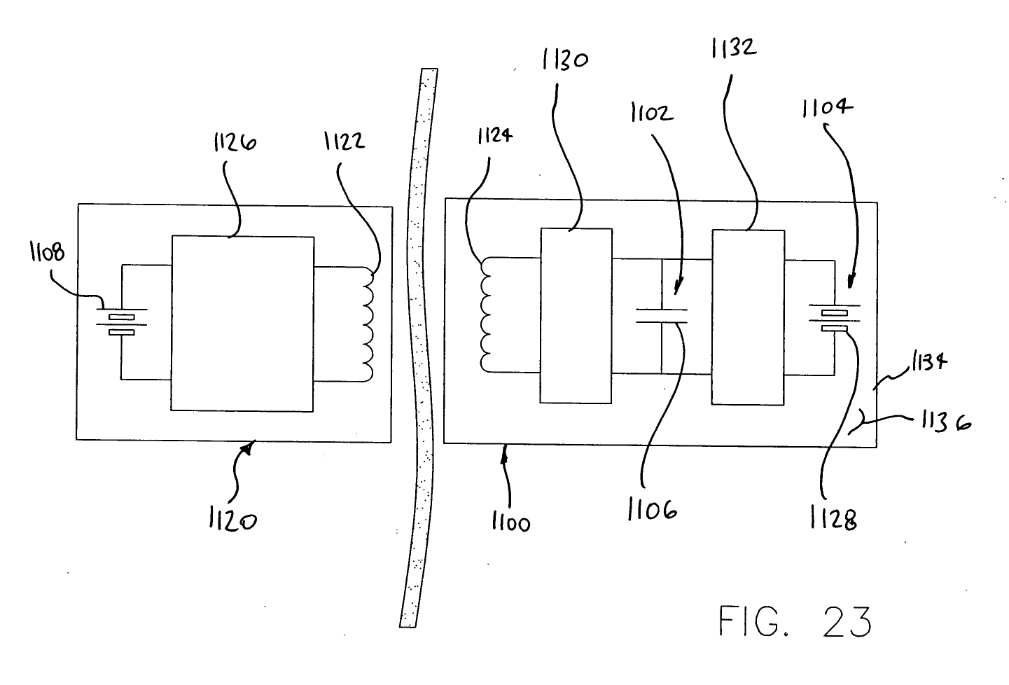

FIG. 23 is a block diagram showing an implantable medical device 1100 and a recharging device 1120 that may be used to recharge implantable medical device 1100. In the embodiment of FIG. 23, recharging device 1120 comprises a first coil 1122 and a first battery 1108 coupled to first coil 1122 for exciting first coil 1122. A control circuit 1126 is connected between first coil 1122 and first battery 1108. Control circuit 1126 is capable of generating the oscillating current necessary to inductively couple first coil 1122 of recharging device 1120 with a second coil 1124 of implantable medical device 1100.

Implantable medical device 1100 comprises a first energy storage element 1102 and a second energy storage element 1104. In the embodiment of FIG. 23, first energy storage element 1102 comprises a capacitor 1106 and second energy storage element 1104 comprises a second battery 1128. A second coil 1124 and a first regulator 1130 are connected to first energy storage element 1102.

In the embodiment of FIG. 23, second coil 1124 and first regulator 1130 can cooperate to charge first energy storage element 1102. First regulator 1130 is capable of controlling the flow of current and the magnitude of voltage applied to first energy storage element so that first energy storage element 1102 is charged at a first charging rate. First regulator 1130 may comprise, for example, a current regulator and/or a voltage regulator.

In the embodiment of FIG. 23, a second regulator 1132 is interposed between the first energy storage element 1102 and second energy storage element 1104. Second regulator 1132 is capable of controlling the flow of current and the magnitude of voltage applied to second energy storage element so that second energy storage element 1104 is charged at a second charging rate. Second regulator 1132 may comprise, for example, a current regulator and/or a voltage regulator.

In the embodiment of FIG. 23, capacitor 1106 is capable of being charged at a faster rate than battery 1108. Accordingly, the second charging rate is slower than the first charging rate. Although one capacitor 1106 is illustrated in FIG. 23, it will be appreciated that embodiments are possible in which capacitor 1106 comprises a plurality of capacitors.

As shown in FIG. 23, implantable medical device 1100 comprises a housing 1134 defining a cavity 1136. Housing 1134 may comprise various materials without deviating from the spirit and scope of the present invention. Examples of materials that may be suitable in some applications include titanium and stainless steel. With reference to FIG. 23, it will be appreciated that second coil 1124, first regulator 1130 and first battery 1108 are disposed in cavity 1136 defined by housing 1134.

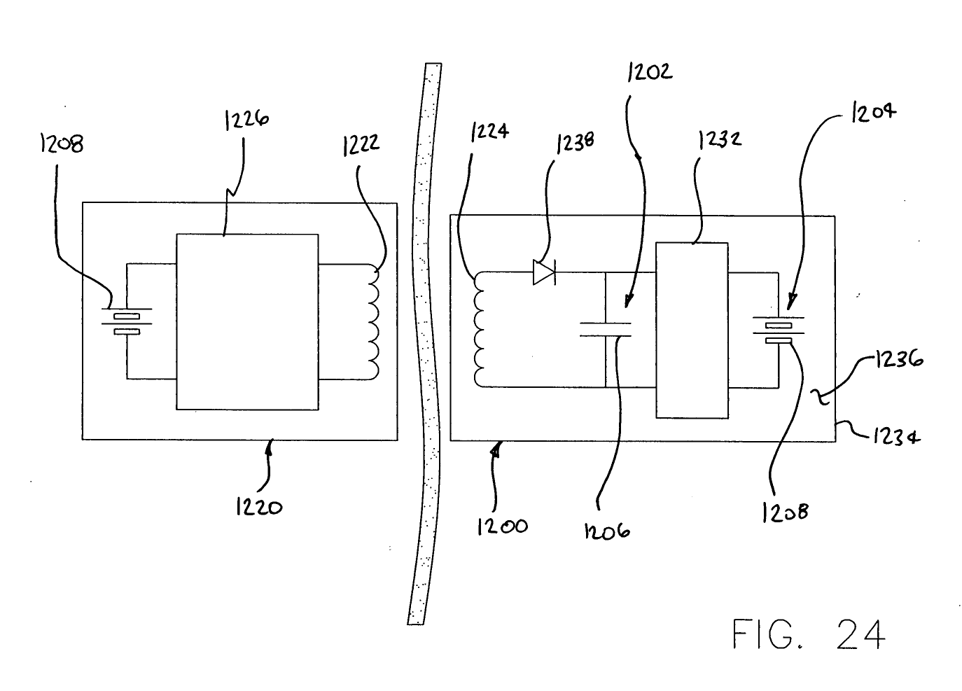

FIG. 24 is a block diagram showing an implantable medical device 1200 and a recharging device 1220 that may be used to recharge implantable medical device 1200. In the embodiment of FIG. 24, recharging device 1220 comprises a first coil 1222 and a first battery 1208 coupled to first coil 1222 for exciting first coil 1222. A control circuit 1226 is connected between first coil 1222 and first battery 1208. Control circuit 1226 is capable of generating the oscillating current necessary to inductively couple first coil 1222 of recharging device 1220 with a second coil 1224 of implantable medical device 1200.

Second coil 1224 of implantable medical device 1200 is coupled to a first energy storage element 1202 by a diode 1238. Implantable medical device 1200 also includes a second energy storage element 1204. In the embodiment of FIG. 24, second coil 1224 and diode 1238 can cooperate to charge first energy storage element 1202. In the embodiment of FIG. 24, a regulator 1232 is interposed between the first energy storage element 1202 and second energy storage element 1204. Regulator 1232 is capable of controlling the flow of current and the magnitude of voltage applied to second energy storage element so that second energy storage element 1204 is charged at a controlled charging rate. Regulator 1232 may comprise, for example, a current regulator and/or a voltage regulator.

In the embodiment of FIG. 24, first energy storage element 1202 comprises a capacitor 1206 and second energy storage element 1204 comprises a battery 1208. In this embodiment, capacitor 1206 is capable of being charged at a faster rate than battery 1208. Accordingly, regulator 1232 may be used to charge battery 1208 at a second charging rate is slower than a first charging rate that capacitor 1206 is capable of. Although one capacitor 1206 is illustrated in FIG. 24, it will be appreciated that embodiments are possible in which capacitor 1206 comprises a plurality of capacitors.

As shown in FIG. 24, implantable medical device 1200 comprises a housing 1234 defining a cavity 1236. Housing 1234 may comprise various materials without deviating from the spirit and scope of the present invention. Examples of materials that may be suitable in some applications include titanium and stainless steel. With reference to FIG. 24, it will be appreciated that second coil 1224, first regulator 1230 and first battery 1208 are disposed in cavity 1236 defined by housing 1234.

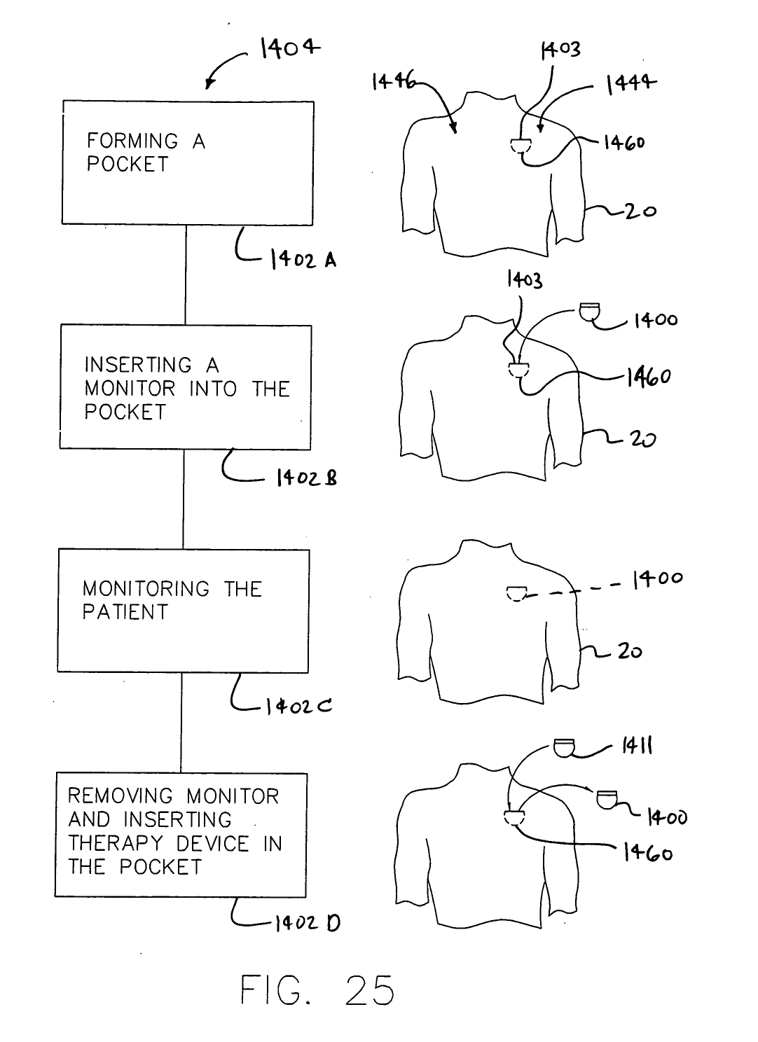

FIG. 25 shows a flowchart 1404 illustrating an exemplary method in accordance with the present invention. Block 1402A of flowchart 1404 illustrates the step of forming a pocket 1460 in a left implant site 1444 in the body of a patient 20. In should be noted that pocket 1460 may be formed in a right implant site 1446 of the body of patient 20 without deviating from the spirit and scope of the present invention. Pocket 1460 may be formed, for example, by making an incision 1403 with a cutting tool and pushing a blunt object through the incision 1403 to displace tissue and form pocket 1460. Pocket 1460 may also be formed by pushing gloved fingers through incision 1403.

Block 1402B of flowchart 1404 illustrates the step of inserting an implantable monitoring device 1400 in pocket 1460. Implantable monitoring device may comprise, for example, the implantable medical devices described herein. Implantable monitoring device 1400 may be inserted through incision 1403 so that the housing of implantable monitoring device 1400 is positioned within pocket 1460 adjacent to incision 1403. Incision 1403 may then be closed and the patient may be allowed to go about a normal daily routine.

Block 1402C of flowchart 1404 illustrates the step of monitoring the patient. Implantable monitoring device 1400 may detect various physiological parameters such as, for example, ECG, pressure and temperature. Implantable monitoring device 1400 may transmit (e.g., wirelessly) signals related to these parameters to a repeater worn by or kept near patient 20. Patient 20 may be monitored during normal daily activity for a period of weeks, months and/or years.

A method in accordance with the present invention may include, for example, the steps of placing an implantable monitoring device comprising a conductive housing and a remote electrode in a left implant site 1444 and detecting a voltage difference between the remote electrode and the conductive housing. This method may further include the step of producing a signal representative of the voltage difference between the remote electrode and the conductive housing. The signal may be transmitted to a receiver located outside the human body. Information obtained during the monitoring step may be analyzed to determine what type of implantable therapy device may be appropriate for patient 20.

Block 1402D of flowchart 1404 illustrates the steps of removing implantable monitoring device 1400 from pocket 1460 and inserting an implantable therapy device 1411 in pocket 1460. In some useful methods in accordance with the present invention, implantable monitoring device 1400 is removed from pocket 1460 and implantable therapy device 1411 is inserted in pocket 1460 during a single surgical procedure. In the embodiment of FIG. 25, implantable monitoring device 1400 and implantable therapy device 1411 have similar shapes and a similar in size.

Implantable therapy device 1411 may comprise various elements without deviating from the spirit and scope of the present invention. Examples of implantable therapy devices that may be suitable in some applications include pacemakers, defibrillators, and/or cardioverters. In some useful methods in accordance with the present invention, pocket 1460 is disposed in a location which will allow leads connected to implantable therapy device 1411 to travel through the vasculature of patient 20 to the heart of patient 20.

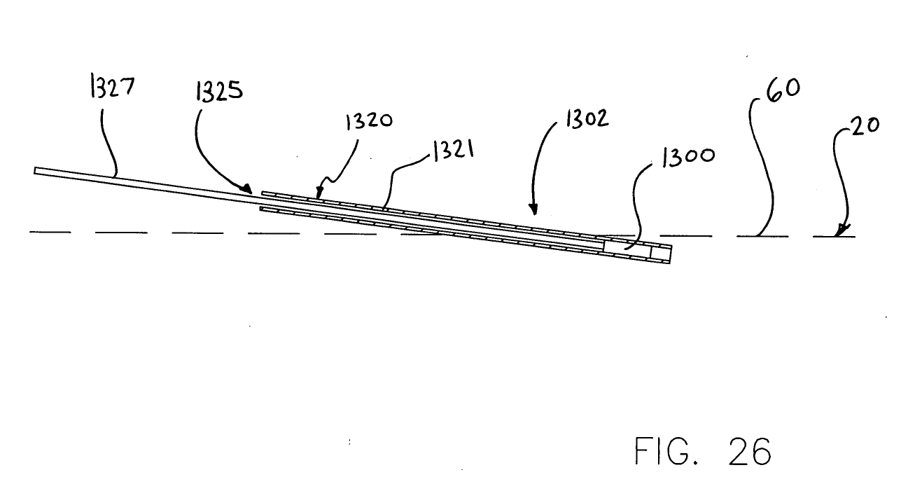

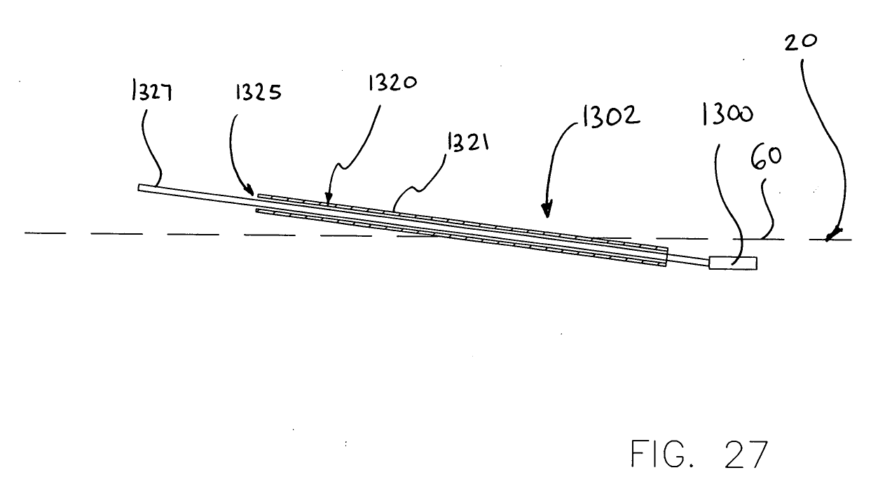

FIGS. 26 and 27 are diagram views showing a placement tool 1320 that may be employed to deploy an implantable medical device 1300 in accordance with the present invention. Placement tool 1320 comprises a wall 1321 defining a lumen 1325. A shaft 1327 has been inserted into the lumen 1325 of placement tool 1320. To implant implantable medical device 1300, an incision 1302 is made where the device is to be inserted under the skin 60 of patient 20. Placement tool 1320 is directed through the incision 1302 and under the skin 60 until the distal end of placement tool 1320 is proximate a desired location for implantable medical device 1300. Shaft 1327 is moved distally so that implantable medical device 1300 exits the distal end of placement tool 1320. Placement tool 1320 may then be extracted, leaving implantable medical device 1300 in the desired position.

It should be recognized to those skilled in the art that the devices described here can be applied for monitoring of other physiological signals such as those which can be measured on or within the heart, brain, bladder, transplanted organs, arteries, veins, and other body tissues.

Those skilled in the art will recognize that the present invention may be manifested in a variety of forms other than the specific embodiments described herein. Accordingly, departures in form and detail may be made without departing from the spirit and scope of the present invention as described in the appended claims.

1. A method comprising the steps of:
forming a pocket in an implant site overlaying a rib cage of a human body;
inserting an implantable monitoring device into the pocket;
removing the implantable monitoring device from the pocket; and
inserting an implantable therapy device into the pocket.

2. The method of claim 1, wherein the implantable monitoring device has a size and shape that is similar to a size and shape of the implantable therapy device.

3. The method of claim 2, further including the steps of:
detecting a voltage difference between a conductive housing of the heart monitor and a remote electrode of the heart monitor.
producing a signal representative of the voltage difference between the remote electrode and the conductive housing; and
transmitting the signal to a receiver located outside the human body.

4. The method of claim 1, wherein the implant site extends between a skin and a rib cage of the human body.

5. The method of claim 1, wherein the implant site extends between a left-most extent of a sternum of the human body and a left-most extent of a rib cage of the human body.

6. The method of claim 1, wherein the implant site extends between a right-most extent of a sternum of the human body and a right-most extent of a rib cage of the human body.

7. The method of claim 1, wherein the implant site extends between a lower-most surface of a clavicle of the human body and a lower-most extent of a sternum of the human body.

8. The method of claim 1, wherein the heart monitor is placed between a skin and a rib cage of the human body.

9. The method of claim 1, wherein the heart monitor is placed between a left-most extent of a sternum of the human body and a left-most extent of a rib cage of the human body.

10. The method of claim 1, wherein the heart monitor is placed between a right-most extent of a sternum of the human body and a right-most extent of a rib cage of the human body.

11. The method of claim 1, wherein the heart monitor is placed between a lower-most surface of a clavicle of the human body and a lower-most extent of a sternum of the human body.

12 A method comprising the steps of:
placing an implantable medical device comprising a conductive housing and a remote electrode in an implant site proximate a left arm of a human body;
detecting a voltage difference between the remote electrode and the conductive housing;
producing a signal representative of the voltage difference between the remote electrode and the conductive housing; and
transmitting the signal to a receiver located outside the human body.

13. The method of claim 12, wherein the step of placing the implantable medical device in the implant site comprises the steps of:
forming a pocket in the implant site; and
inserting the conductive housing into the pocket.

14. The method of claim 13, further including the steps of:
removing the implantable medical device from the pocket; and
inserting a heart therapy device into the pocket.

15. The method of claim 12, wherein the step of placing the remote electrode and the conductive housing in the implant site comprises the steps of:
forming a pocket in the implant site;
forming a channel in the implant site such that the channel communicates with the pocket;
placing the remote electrode in the channel;
connecting the remote electrode to the conductive housing; and
placing the conductive housing in the pocket.

16. The method of claim 12, wherein the implant site extends between a skin and a rib cage of the human body.

17. The method of claim 12, wherein the implant site extends between a left-most extent of a sternum of the human body and a left-most extent of a rib cage of the human body.

18. The method of claim 12, wherein the implant site extends between a right-most extent of a sternum of the human body and a right-most extent of a rib cage of the human body.

19. The method of claim 12, wherein the implant site extends between a lower-most surface of a clavicle of the human body and a lower-most extent of a sternum of the human body.

20. The method of claim 12, wherein the remote electrode and the conductive housing are both placed between a skin and a rib cage of the human body.

21. The method of claim 12, wherein the remote electrode and the conductive housing are both placed between a left-most extent of a sternum of the human body and a left-most extent of a rib cage of the human body.

22. The method of claim 12, wherein the remote electrode and the conductive housing are both placed between a right-most extent of a sternum of the human body and a right-most extent of a rib cage of the human body.

23. The method of claim 12, wherein the remote electrode and the conductive housing are both placed between a lower-most surface of a clavicle of the human body and a lower-most extent of a sternum of the human body.

24. The method of claim 12, wherein the remote electrode and the conductive housing are placed so that the remote electrode and the conductive housing are separated by a center-to center distance that is pre-selected for fitting the remote electrode and the conductive housing within the implant site.

25. The method of claim 24, wherein the pre-selected distance is greater than about 4.0 centimeters and less than about 10.0 centimeters.

26. The method of claim 25, wherein the pre-selected distance is greater than about 5.0 centimeters and less than about 7.0 centimeters.

27. The method of claim 12, wherein the remote electrode and the conductive housing are placed so that the remote electrode and the conductive housing define an overall length of the implantable medical device that is pre-selected for fitting the remote electrode and the conductive housing within the implant site.

28. The method of claim 27, wherein the overall length is greater than about 4.0 centimeters and less than about 13.0 centimeters.

29. The method of claim 25, wherein the overall length is greater than about 5.0 centimeters and less than about 10.0 centimeters.

30. The method of claim 12, further comprising the steps of receiving the transmitted signal and re-transmitting the signal.

31. The method of claim 30, further comprising the steps of receiving the re-transmitted signal and communicating the signal over a network.

32. The method of claim 31, wherein the network comprises the internet.

33. The method of claim 31, further including the step of analyzing the signal.

34. The method of claim 33, wherein the signal is analyzed after the signal is communicated over the network.

35. The method of claim 12, wherein the signal is transmitted substantially in real time.

36. The method of claim 12, wherein the signal transmitted at a given point in time is representative of the voltage difference being detected at the given point time.

37. The method of claim 12, wherein the signal is a function of the voltage difference between the remote electrode and the conductive housing.

38. The method of claim 12, wherein the step of producing a signal representative of the voltage difference between the remote electrode and the conductive housing comprises the step of amplifying the voltage difference between the remote electrode and the conductive housing.

39. The method of claim 12, further comprising the step of filtering the signal.

40. An implantable medical device, comprising:
a conductive housing;
a remote electrode mechanically coupled to the conductive housing by a lead body;
an amplifier electrically connected to the remote electrode and the conductive housing for providing a signal representative of a voltage difference between the remote electrode and the conductive housing.

41. The implantable medical device of claim 40, wherein further including a battery disposed in the conductive housing.

42. The implantable medical device of claim 41, wherein further including a battery disposed in the conductive housing.

43. The implantable medical device of claim 42, wherein the implantable power source comprises a means for generating power from a human body.

44. The implantable medical device of claim 40, wherein the lead body separates remote electrode and the conductive housing by a center to center distance that is selected so that the conductive housing, the remote electrode, and the lead body will all be received in an implant site overlaying one half of a rib cage of a human body.

45. The implantable medical device of claim 44, wherein the implant site extends between a skin and a rib cage of the human body.

46. The implantable medical device of claim 44, wherein the implant site extends between a left-most extent of a sternum of the human body and a left-most extent of a rib cage of the human body.

47. The implantable medical device of claim 44, wherein the implant site extends between a right-most extent of a sternum of the human body and a right-most extent of a rib cage of the human body.

48. The implantable medical device of claim 44, wherein the implant site extends between a lower-most surface of a clavicle of the human body and a lower-most extent of a sternum of the human body.

49. The implantable medical device of claim 44, wherein the pre-selected distance is greater than about 4.0 centimeters and less than about 10.0 centimeters.

50. The implantable medical device of claim 44, wherein the pre-selected distance is greater than about 5.0 centimeters and less than about 7.0 centimeters.

51. The implantable medical device of claim 40, wherein the remote electrode is detachably attached to the conductive housing by the lead body.

52. The implantable medical device of claim 40, further comprising a transmitter for transmitting a signal produced by the amplifier.

53. The implantable medical device of claim 40, wherein the lead body has sufficient lateral flexibility to allow relative motion between the electrode and the conductive housing in response to muscle movements of the human body.

54. The implantable medical device of claim 40, wherein the lead body has sufficient longitudinal stiffness to maintain spacing between the electrode and the conductive housing.

55. The implantable medical device of claim 40, wherein the remote electrode has a generally circular lateral cross section.

56. The implantable medical device of claim 40, wherein the remote electrode comprises a generally cylindrical body portion.

57. The implantable medical device of claim 40, wherein the remote electrode comprises a rounded tip portion.

58. The implantable medical device of claim 57, wherein the remote electrode comprises a generally hemispherical tip portion.

59. The implantable medical device of claim 40, wherein the remote electrode is free of anchors.

60. The implantable medical device of claim 40, wherein the lead body is free of anchors.

61. In combination:
a charging device comprising a first coil and a first battery coupled to the first coil for exciting the first coil;
an implantable medical device comprising a second battery and a second coil coupled to the second battery for charging the second battery;
the first coil and the second coil being inductively coupled to one another so that the second battery is charged while the first battery is depleted.

62. The combination of claim 61, wherein the implantable medical device is disposed inside a human body and the charging device is disposed outside of the human body.

63. The combination of claim 61, wherein the charging device comprises a housing defining a cavity.

64. The combination of claim 63, wherein the first battery is disposed within the cavity defined by the housing of the charging device.

65. The combination of claim 61, wherein the first battery is capable of satisfying the power requirements of the charging device.

66. The combination of claim 61, wherein the first battery is larger than the second battery.

67. The combination of claim 66, wherein the first battery is sufficiently larger than the second battery so that the first battery has sufficient capacity for fully charging the second battery and compensating for energy lost during the charging of the second battery.

68. The combination of claim 61, wherein the first battery is the sole source of power for the charging device.

69. The combination of claim 61, further comprising a garment for holding the charging device proximate the implanted medical device.

70. The combination of claim 69, wherein the garment comprises a pocket that is dimensioned to receive the charging device.

71. An implantable medical device, comprising:
a first energy storage element;
a first coil and a first regulator coupled to the first energy storage element for charging the first energy storage element at a first charging rate;
a second energy storage element coupled to the first energy storage element; and
a second regulator interposed between the first energy storage element and the second energy storage element for charging the second energy storage element at a second charging rate.

72. The implantable medical device of claim 71, wherein the second charging rate is different from the first charging rate.

73. The implantable medical device of claim 72, wherein the second charging rate is slower than the first charging rate.

74. The implantable medical device of claim 71, wherein the first energy storage element comprises one or more capacitors.

75. The implantable medical device of claim 71, further comprising a housing defining a cavity.

76. The implantable medical device of claim 75, wherein the housing comprises a metallic material.

77. The implantable medical device of claim 76, wherein the metallic material comprises titanium.

78. The implantable medical device of claim 75, wherein the first coil is disposed in the cavity defined by the housing.

79. The implantable medical device of claim 71, wherein the second energy storage element comprises a battery.

80. An implantable medical device, comprising:
a first energy storage element;
a coil coupled to the first energy storage element for charging the first energy storage element;
a second energy storage element coupled to the first energy storage element; and
a regulator interposed between the first energy storage element and the second energy storage element for charging the second energy storage element at a second charging rate.

81. The implantable medical device of claim 80, further including a diode connected between the coil and the first energy storage element.

82. The implantable medical device of claim 80, wherein the first energy storage element comprises one or more capacitors.

83. The implantable medical device of claim 80, further comprising a housing defining a cavity.

84. The implantable medical device of claim 83, wherein the housing comprises a metallic material.

85. The implantable medical device of claim 85, wherein the metallic material comprises titanium.

86. The implantable medical device of claim 83, wherein the first coil is disposed in the cavity defined by the housing.

87. The implantable medical device of claim 80, wherein the second energy storage element comprises a battery.

88. A method for placing an implantable medical device in a body, comprising the steps of:
providing an implantable medical device disposed in a lumen defined by a placement tool;
inserting a distal end of the placement tool into the body; and
moving a shaft distally within the lumen of the placement tool for urging the implantable medical device to exit the distal end of the placement tool.

89. In combination:
a placement tool having a proximal end, a distal end, and a lumen extending therebetween;
a shaft disposed in sliding engagement with the lumen of the placement tool;
the shaft being partially disposed within the lumen of the placement tool; and
an implantable medical device disposed in the lumen of the delivery tool.

90. The combination of claim 89, wherein a distal end of the shaft is disposed in the lumen of the placement tool and a proximal end of the shaft is disposed outside of the lumen of the placement tool.

91. The combination of claim 89, wherein the implantable medical device is disposed proximate a distal end of the placement tool.

92. The combination of claim 89, wherein the implantable medical device is disposed proximate a distal end of the shaft.

93. The combination of claim 92, wherein a distal end of the shaft contacts the implantable medical device.

94. The combination of claim 89, wherein the implantable medical device comprises a first electrode, a second electrode, and an amplifier electrically connected to the first electrode and the second electrode for providing a signal representative of a voltage difference between the first electrode and the second electrode.

95. The combination of claim 94, wherein the first electrode and the second electrode are capable of contacting a tissue of a body.

96. The combination of claim 89, wherein the implantable medical device is capable of detecting a physiological parameter of a human body.

97. The combination of claim 96, wherein the physiological parameter is selected from the group consisting of ECG, pressure, patient activity, patient posture, impedance, respiratory rate, respiratory effort, and glucose.
